(19) United States
(12) Patent Application Publication
(10) Pub. No.: US 2020/0323815 A1
(43) Pub. Date: Oct. 15, 2020

(54) AMORPHOUS DISPERSIONS OF EPIGALLOCATECHIN GALLATE

(71) Applicant: AMRI SSCI, LLC, West Lafayette, IN (US)

(72) Inventors: Jing TENG, West Lafayette, IN (US); Yizheng CAO, West Lafayette, IN (US); Jon Gordon SELBO, West Lafayette, IN (US)

(21) Appl. No.: 16/632,658

(22) PCT Filed: Jul. 20, 2018
(86) PCT No.: PCT/US18/43107

§ 371 Date: Jan. 21, 2020

Related U.S. Application Data
(60) Provisional application No. 62/535,075, filed on Jul. 20, 2017.

(51) Int. Cl. A61K 31/353; A61K 47/34; A61K 47/38; A23L 29/262; A23L 33/105; A23L 33/24

(52) U.S. Cl. CPC A61K 31/353; A61K 47/34; A61K 47/38; A61K 9/20; A23L 33/105; A23L 33/24; A23L 29/262

(57) ABSTRACT

Amorphous dispersions of epigallocatechin gallate are herein described. In addition, pharmaceutical compositions comprising such dispersions and methods of treating diseases with such dispersions and pharmaceutical compositions are also described.

This application claims priority benefit of U.S. Provisional Patent Application Ser. No. 62/535,075 filed Jul. 20, 2017, which is hereby incorporated by reference in its entirety.

### BACKGROUND OF THE INVENTION

Green tea has been one of the most commonly consumed beverages since ancient times (Cabrera, C., et al., 2006). The benefits of green tea include antiarthritic, antibacterial, antiangiogenic, and antioxidative properties (Chowdhury, A., et al., 2016). The benefits are attributed to the polyphenols, of which catechin is the major component, including epicatechin (EC), epigallo-catechin (EGC), epicatechin-3-gallate (ECG), and epigallocatechin gallate (EGCG). Catechins are widely considered a preventive agent against mammary cancer post-initiation, degenerative diseases, oxidative stress, cardiovascular and neurological disorders, and hepatotoxicity (Chowdhury, et al., 2016). Many of these beneficial health effects are credited to the most abundant catechin: epigallocatechin gallate (“EGCG”) (Mandel, S., et al., 2004; Moyers, S. B., et al., 2004). FIG. 1 summarizes the commonly used hierarchical nomenclature to describe “green tea extracts”.

As the most abundant catechin, EGCG accounts for 60-70% of total tea catechins (Katiyar, S., et al., 1996), is often used as a quality indicator (Ananingsih, V. K., et al., 2013), and is claimed to be the most prominent catechin for health benefit purposes (Khan, N., et al., 2007). Due to its strong antioxidant and cancer chemopreventive properties, it is also one of the most extensively explored polyphenolic components of green tea (Kim, H.-S., et al., 2014; Staszewski, M.v., et al., 2011; Du, G.-J., et al., 2012; Singh, B. N., et al., 2011). EGCG is also the only polyphenol present in plasma at high concentration (77-90%) in free form (Manach, C., et al., 2005).

To date, EGCG has been demonstrated to be an anticancer agent (Chung, S., et al., 2015; Ho, Y.-C., et al., 2007; Shankar, S., et al., 2008), antioxidant, and antibacterial agent (Shutava, T. G., et al., 2009), with chemopreventive, anti-inflammatory, and anti-aging properties (Hu, C., et al., 2016). It is also reported to be protective against cardiovascular disease (Cai, Y., et al., 2013; Hong, et al., 2014; Zeng, X., et al., 2015), neurodegenerative diseases (He, M., et al., 2012; Lorenzen, N., et al., 2014), UV-induced photodamage, basal cell carcinomas, melanomas, skin papillomas (Katiyar, S. K., et al., 2000; Wang, Z. Y., et al., 1992), obesity, and diabetes (Chowdhury, et al., 2016). In addition, EGCG has been shown to interact with a number of proteins such as α-synuclein, amyloid-β, and huntingtin (Hora, M., et al., 2017). The structure of EGCG is:

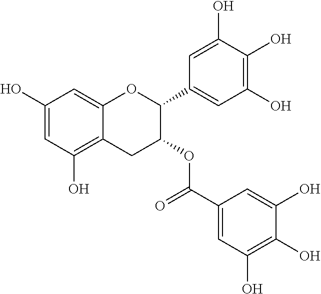 [SMILES: O=C(O[C@@H]1Cc2c(O)cc(O)cc2O[C@@H]1c1cc(O)c(O)c(O)c1)c1cc(O)c(O)c(O)c1]

While the vast potential of EGCG in health care has been recognized, two major obstacles have hindered its utilization: high instability and low bioavailability. The instability of green tea catechins has been under study for several decades (Ananingsih, V. K., et al., 2013). EGCG has shown considerable instability and degradability in both the solid state and in solutions (Li, N., et al., 2013; Li, N., et al., 2014). A cow study found that catechins including EGCG are substantially degraded by rumen microorganisms resulting in no detectable catechin in plasma. However intraduodenal administration improved plasma concentration of all catechins with increasing dosage (Wein, S., et al., 2016). The instability of EGCG has crucially affected its processing, storage (Ananingsih, V. K., et al., 2013), and, as expected, dosing in the gastrointestinal (GI) tract. Effects of EGCG take place in a plasma level dependent manner (Mereles, D., et al., 2011) and the necessary plasma concentration, is believed to be relatively high (Smith, A. J., et al., 2013). In the meantime, the poor bioavailability of EGCG has been reported in rodents (Chen, L., et al., 1997) and humans (Chow, H.-H. S., et al., 2001), with values as low as 2-5% (Catterall, et al., 2003; Patel, A. R., et al., 2011b). Although factors responsible for the poor bioavailability of EGCG have not been fully understood, the instability/degradation of EGCG in the GI tract and its rapid in vivo dissolution and elimination (Cai, Y., et al., 2002; Dvorakova, K., et al., 1999; Manach, C., et al., 2005) are believed to be important causes. The reported elimination half-life of EGCG is 3.4 h±0.3 h (Lee, M.-J., et al., 2002).

A commonly hypothesized approach to improve bioavailability is to decrease the dissolution rate and therefore establish an extended residence and absorption of EGCG in the GI tract (Smith, A., et al., 2010). An improvement of 30% in bioavailability has been reported with coadministration of piperine, an alkaloid from black pepper, as a result of the extended GI transit, allowing for longer residence time in the intestine (Lambert, J. D., et al., 2004). However, consumption of piperine may influence the metabolism of food and drugs, bringing possible negative effects (Smith, A., Giunta, et al., 2010). Methods such as encapsulation or making insoluble complexes (Patel, A. R., et al., 2011a; Patel, A. R., et al., 2011b) might result in a slower release of EGCG from the capsulated structure/complex which also diminishes its chemical degradation in the GI tract. However, release data were not reported for pure EGCG and it is known that the manufacturing properties of such complexes are challenging to control. Indeed, a layer-by-layer assembly to encapsulate EGCG with gelatin has been done (Shutava, T. G., et al., 2009) and a fabricated colloidal EGCG-methylcellulose complexes in aqueous suspensions resulting in a sustained release spanning two hours in both simulated intestinal and gastric fluids has also been attempted (Patel, A. R., et al., 2011b). However, the release data were not compared with pure EGCG and results after the first two hours were not provided. The oral bioavailability with a nanolipidic formulation was doubled in one report, which however involved an alcohol suspension, and therefore limited the application (Smith, A., et al., 2010). In a later work, the same group attempted to generate different forms of EGCG cocrystals to lower aqueous solubility (Smith, A. J., et al., 2013), whereas it was found that merely decreasing solubility by up to one order of magnitude was not effective in enhancing bioavailability. There remains, therefore, a need to provide stable and bioavailable EGCG.

### SUMMARY OF THE INVENTION

In one aspect of the invention, a solid dispersion comprising amorphous EGCG and a polymer is provided.

In another aspect of the invention, a pharmaceutical composition comprising a solid dispersion of amorphous EGCG is provided.

In a further aspect of the invention, a method of sustained release delivery of EGCG comprising administering a pharmaceutical composition comprising a solid dispersion of amorphous EGCG is provided.

In yet another aspect of the invention, a sustained release pharmaceutical composition comprising a solid dispersion of amorphous EGCG is provided.

In an additional aspect of the invention, a solid dispersion comprising EGCG and a micelle-forming polymer is provided.

In another aspect of the invention, a tablet or capsule comprising solid dispersions of EGCG and polymers are provided.

In a further aspect of the invention, a foodstuff additive comprising a solid dispersion of EGCG and a polymer is provided.

### BRIEF DESCRIPTION OF THE FIGURES

FIG. 1 is a hierarchical terminology of green tea extracts.

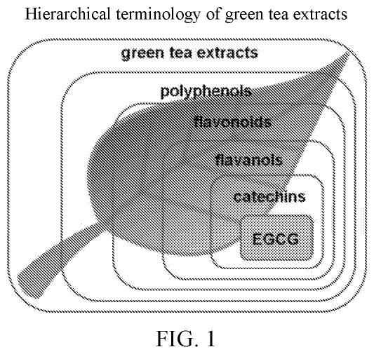

FIG. 2 is an x-ray powder diffraction pattern of crystalline EGCG (cEGCG) isolated from Teavigo®.

FIG. 3 is a set of scanning electron micrographs of cEGCG.

FIG. 4 is a thermogravimetric analysis thermogram of cEGCG.

FIG. 5 is a release profile of cEGCG in pH 7.4 Phosphate-buffered saline (PBS) medium at 37° C.

FIG. 6 is an x-ray powder diffraction pattern of lyopholized amorphous EGCG (aEGCG)

FIG. 7 is a set of scanning electron micrographs of aEGCG.

FIG. 8 is a thermogravimetic analysis thermogram of aEGCG.

FIG. 9 is a modulated differential scanning calorimetry thermogram of aEGCG.

FIG. 10 is an x-ray powder diffraction pattern of aEGCG which has been stressed at 11 days at 40° C./75% relative humidity.

FIG. 11 is the release profile of aEGCG in pH 7.4 PBS medium at 37° C.

FIG. 12 is an x-ray powder diffraction pattern of an EGCG/HPMC-AS dispersion

FIG. 13 is a set of scanning electron micrographs of an EGCG/HPMC-AS dispersion.

FIG. 14 is a thermogravimetric thermogram of an EGCG/HPMC-AS dispersion.

FIG. 15 is a modulated differential scanning calorimetry thermogram of an EGCG/HPMC-AS dispersion.

FIG. 16 is an x-ray powder diffraction pattern of an EGCG/HPMC-AS dispersion which has been stressed at 11 days at 40° C./75% relative humidity.

FIG. 17 is the release profile of an EGCG/HPMC-AS dispersion in pH 7.4 PBS medium at 37° C.

FIG. 18 is an x-ray powder diffraction pattern of an EGCG/HPMC-P dispersion.

FIG. 19 is a set of scanning electron micrographs of an EGCG/HPMC-P dispersion.

FIG. 20 is a thermogravimetric thermogram of an EGCG/HPMC-P dispersion.

FIG. 21 is a modulated differential scanning calorimetry thermogram of an EGCG/HPMC-P dispersion.

FIG. 22 is an x-ray powder diffraction pattern of an EGCG/HPMC-P dispersion which has been stressed at 11 days at 40° C./75% relative humidity.

FIG. 23 is a release profile of a dispersion of EGCG/HPMC-P dispersion in pH 7.4 PBS medium at 37° C.

FIG. 24 is an x-ray powder diffraction pattern of an EGCG/Soluplus® dispersion.

FIG. 25 is a set of scanning electron micrographs of an EGCG/Soluplus® dispersion.

FIG. 26 is a thermogravimetric analysis thermogram of EGCG/Soluplus® dispersion.

FIG. 27 is a modulated differential scanning calorimetry thermogram of EGCG/Soluplus® dispersion.

FIG. 28 is an x-ray powder diffraction pattern of EGCG/Soluplus® dispersion which has been stressed at 11 days at 40° C./75% relative humidity.

FIG. 29 is a release profile of a dispersion of EGCG/Soluplus® dispersion in pH 7.4 PBS medium at 37° C.

FIG. 30 is an x-ray powder diffraction pattern of an EGCG/cellulose acetate dispersion.

FIG. 31 is a set of scanning electron micrographs of an EGCG/cellulose acetate dispersion.

FIG. 32 is a thermogravimetric analysis thermogram of an EGCG/cellulose acetate dispersion.

FIG. 33 is a modulated differential scanning thermogram of an EGCG/cellulose acetate dispersion.

FIG. 34 is an x-ray powder diffraction pattern of an EGCG/cellulose acetate dispersion which has been stressed at 11 days at 40° C./75% relative humidity.

FIG. 35 is a release profile of an EGCG/cellulose acetate dispersion in pH 7.4 PBS medium at 37° C.

FIG. 36 is an overlay of x-ray powder diffraction patterns of the four dispersions, aEGCG, and cEGCG.

FIG. 37 is an overlay of thermogravimetric analysis thermograms of the four dispersions, aEGCG, and cEGCG.

FIG. 38 is an overlay of modulated differential scanning calorimetry thermograms of the four dispersions and aEGCG.

FIG. 39 is an overlay of x-ray powder diffraction patterns of the four dispersions and aEGCG after stressing at 11 days at 40° C./75% relative humidity.

FIG. 40 is an overlay of release profiles of the four dispersions, aEGCG, and cEGCG at pH 7.4 PBS medium at 37° C.

FIG. 41 is an x-ray powder diffraction overlay of post-dissolution solids from EGCG/Soluplus® and EGCG/cellulose acetate dispersions.

FIG. 42 is a comparison of EGCG release from the dispersions, and amorphous and crystalline EGCG in the first 20 minutes in pH 7.4 PBS medium at 37° C.

FIG. 43 is a kinetic release model fit for cEGCG.

FIG. 44 is a kinetic release model fit for aEGCG.

FIG. 45 is a kinetic release model fit for EGCG/HPMC-AS.

FIG. 46 is a kinetic release model fit for EGCG/HPMC-P

FIG. 47 is a kinetic release model fit for EGCG/cellulose acetate.

FIG. 48 is a kinetic release model fit for EGCG/Soluplus®.

FIG. 49 is a release profile of EGCG/Soluplus® fitted with a biphasic model.

FIG. 50 is an XRPD pattern for a 91:9 (w/w) EGCG/Soluplus® dispersion.

FIG. 51 is an XRPD pattern for an 83:17 (w/w) EGCG/Soluplus® dispersion.

FIG. 52 is an XRPD pattern for a 67:33 (w/w) EGCG/Soluplus® dispersion.

FIG. 53 is an XRPD pattern for a 50:50 (w/w) EGCG/Soluplus® dispersion.

FIG. 54 is an XRPD pattern for a 9:91 (w/w) EGCG/Soluplus® dispersion.

FIG. 55 is an XRPD pattern for a 91:9 (w/w) EGCG/HPMC-AS dispersion.

FIG. 56 is an XRPD pattern for an 83:17 (w/w) EGCG/HPMC-AS dispersion.

FIG. 57 is an XRPD pattern for a 67:33 (w/w) EGCG/HPMC-AS dispersion.

FIG. 58 is an XRPD pattern for a 91:9 (w/w) EGCG/HPMC-P dispersion.

FIG. 59 is an XRPD pattern for an 83:17 (w/w) EGCG/HPMC-P dispersion.

FIG. 60 is an XRPD pattern for a 67:33 (w/w) EGCG/HPMC-P dispersion.

FIG. 61 is an XRPD pattern for a 91:9 (w/w) EGCG/cellulose acetate dispersion.

FIG. 62 is an XRPD pattern for an 83:17 (w/w) EGCG/cellulose acetate dispersion.

FIG. 63 is an XRPD pattern for a 67:33 (w/w) EGCG/cellulose acetate dispersion.

FIG. 64 is an XRPD pattern for amorphous EGCG.

FIG. 65 is an XRPD pattern for the post-stressing solids of a 91:9 (w/w) EGCG/Soluplus® dispersion at about 40° C./75% RH/30 days.

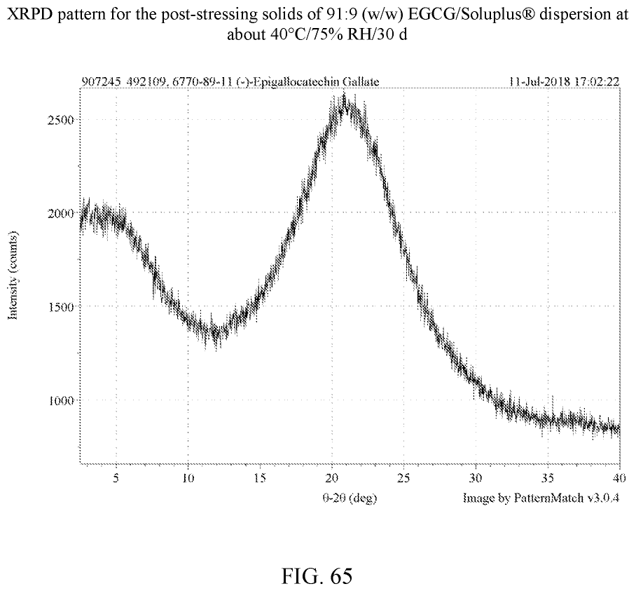

FIG. 66 is an XRPD pattern for the post-stressing solids of an 83:17 (w/w) EGCG/Soluplus® dispersion at about 40° C./75% RH/30 days.

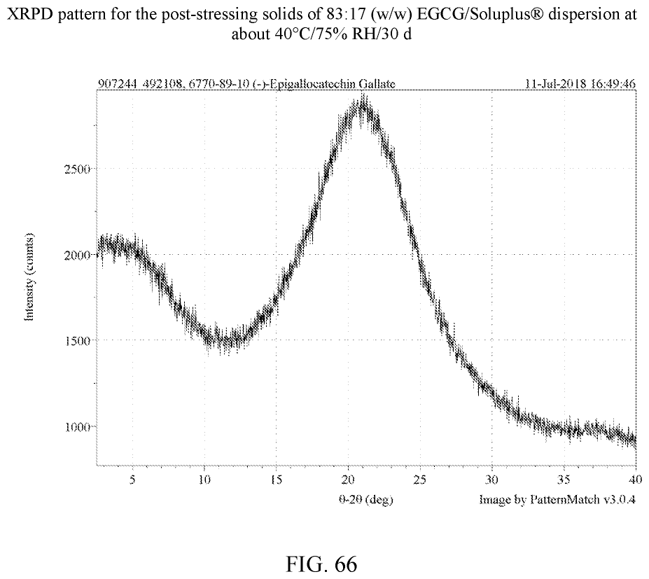

FIG. 67 is an XRPD pattern for the post-stressing solids of a 67:33 (w/w) EGCG/Soluplus® dispersion at about 40° C./75% RH/30 days.

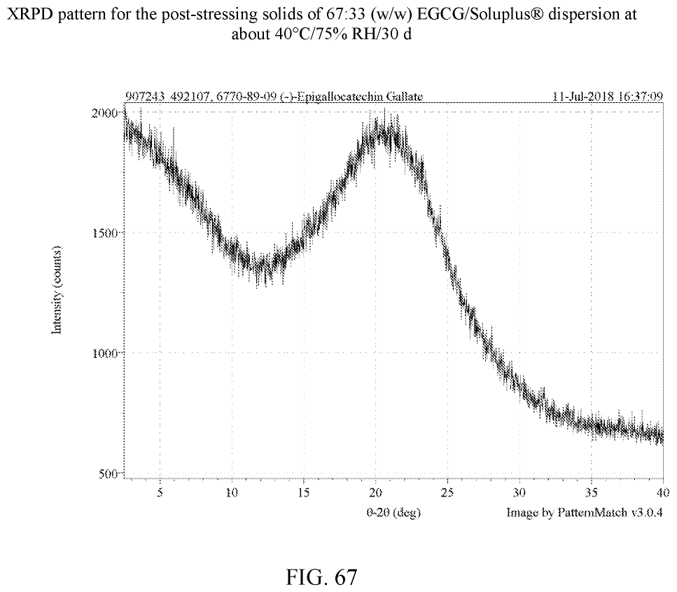

FIG. 68 is an XRPD pattern for the post-stressing solids of a 50:50 (w/w) EGCG/Soluplus® dispersion at about 40° C./75% RH/30 days.

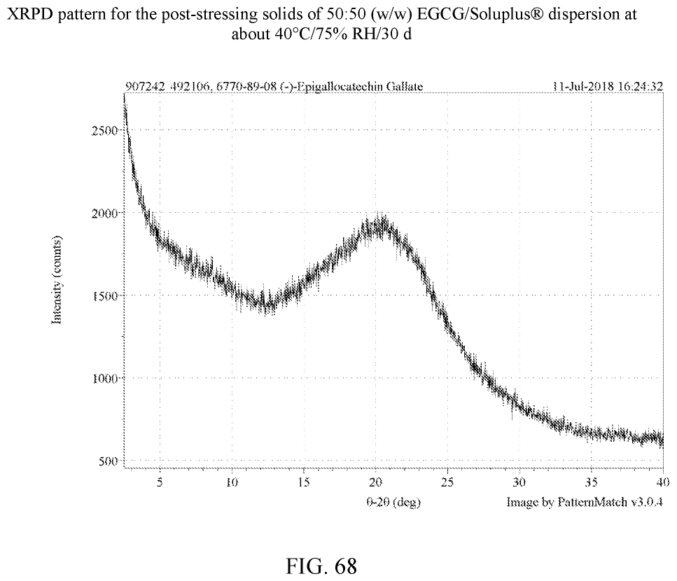

FIG. 69 is an XRPD pattern for the post-stressing solids of a 9:91 (w/w) EGCG/Soluplus® dispersion at about 40° C./75% RH/30 days.

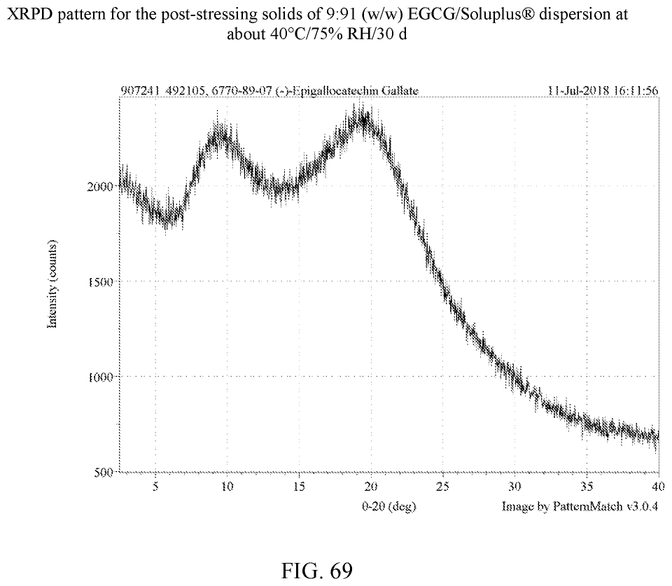

FIG. 70 is an XRPD pattern for the post-stressing solids of a 91:9 (w/w) EGCG/HPMC-AS dispersion at about 40° C./75% RH/30 days.

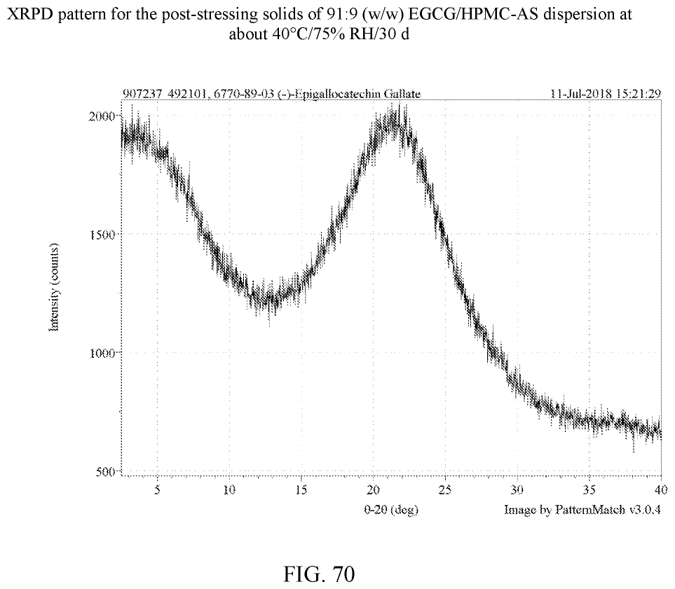

FIG. 71 is an XRPD pattern for the post-stressing solids of an 83:17 (w/w) EGCG/HPMC-AS dispersion at about 40° C./75% RH/30 days.

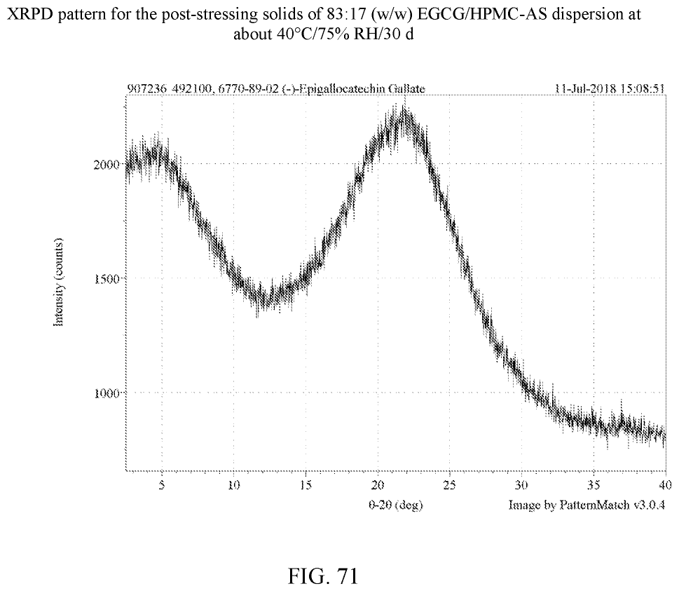

FIG. 72 is an XRPD pattern for the post-stressing solids of a 67:33 (w/w) EGCG/HPMC-AS dispersion at about 40° C./75% RH/30 days.

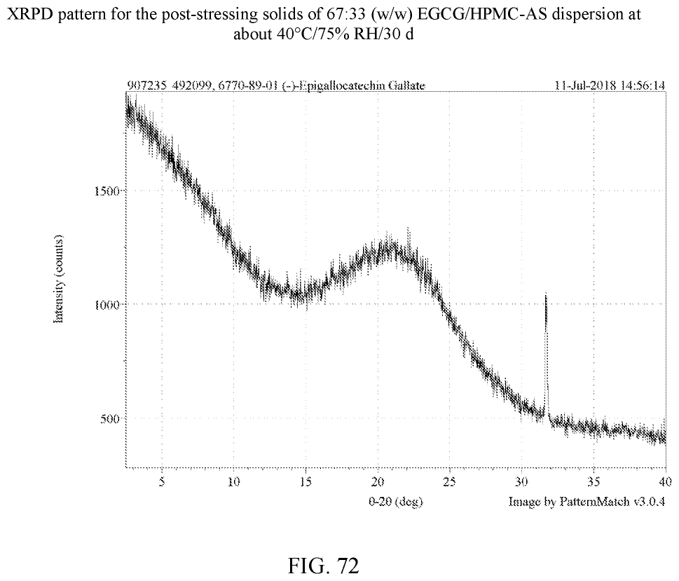

FIG. 73 is an XRPD pattern for the post-stressing solids of a 91:9 (w/w) EGCG/HPMC-P dispersion at about 40° C./75% RH/30 days.

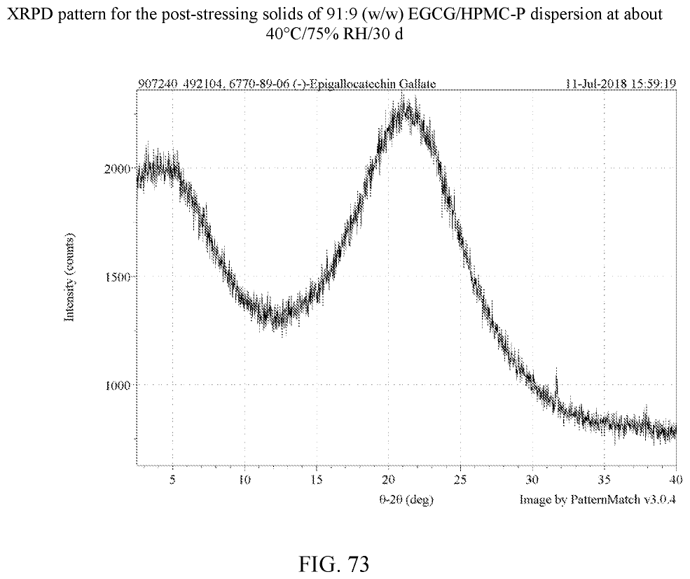

FIG. 74 is an XRPD pattern for the post-stressing solids of an 83:17 (w/w) EGCG/HPMC-P dispersion at about 40° C./75% RH/30 days.

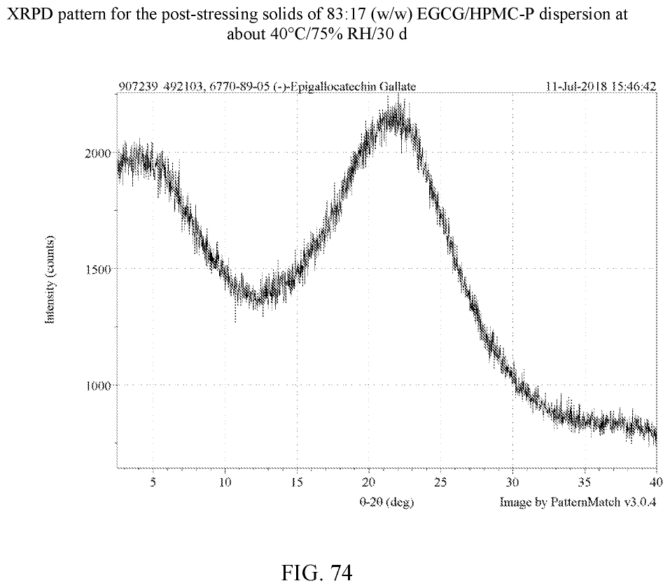

FIG. 75 is an XRPD pattern for the post-stressing solids of a 67:33 (w/w) EGCG/HPMC-P dispersion at about 40° C./75% RH/30 days.

FIG. 76 is an XRPD pattern for the post-stressing solids of a 91:9 (w/w) EGCG/cellulose acetate dispersion at about 40° C./75% RH/30 days.

FIG. 77 is an XRPD pattern for the post-stressing solids of an 83:17 (w/w) EGCG/cellulose acetate dispersion at about 40° C./75% RH/30 days.

FIG. 78 is an XRPD pattern for the post-stressing solids of a 67:33 (w/w) EGCG/cellulose acetate dispersion at about 40° C./75% RH/30 days.

FIG. 79 is an XRPD pattern for the post-stressing solids of amorphous EGCG at about 40° C./75% RH/30 days.

### DETAILED DESCRIPTION OF THE INVENTION

Amorphous materials have been used in the pharmaceutical industry due to their high solubility. However pure amorphous compounds, such as active pharmaceutical ingredients (“APIs”), are rarely marketed as such because of physical instability, i.e., the tendency towards crystallization during storage and processing. Amorphous solid dispersions kinetically stabilize amorphous APIs via the presence of excipients, typically polymers, to help prevent crystallization and maintain the supersaturation (Brouwers, J., Brewster, et al., 2009). Owing to the substantial length and flexibility of the polymer chains in a solid dispersion, an API therein is separated into interstitial solid solutions with the molecules in the interstices. Via this confinement and immobilization, an amorphous API may be significantly stabilized compared to its neat form in the appropriate polymer. As further disclosed herein, Applicants have prepared solid dispersions of amorphous EGCG (where EGCG is the API) and various polymers as vehicles for delivery of EGCG in a pharmaceutical setting. In particular, the amorphous solid dispersions reported herein address the instability and low bioavailability issues which have previously limited the use and development of EGCG.

While the use of solid dispersions to improve the solubility of poorly-soluble drugs is known in the literature, solid dispersions have not, therefore typically been viewed as a vehicle for delivering highly soluble APIs. In this instance, the Applicants have utilized a solid dispersion to provide sustained release formulation with an aim towards enhancing the bioavailability of a water-soluble, but low bioavailability compound, while at the same time addressing the instability challenges plaguing EGCG.

Amorphous dispersions may be made by several methods. These include spray drying, lyophilization, melt extrusion, and co-precipitation. For the solid dispersions prepared in the Examples, lyophilization was used to generate the EGCG amorphous solid dispersions.

The polymers used herein are those that are capable of forming a dispersion with EGCG. While some polymers may not form a dispersion with lyophilziation, such polymers may form a dispersion under different methods or under different lyophilziation conditions than those herein described. Examples of suitable EGCG:polymer weight ratios include about 10:90 to about 90:10 including all ranges in between such as about 10:90 to about 20:80, about 20:80 to about 30:70, about 30:70 to about 40:60, about 40:60 to about 50:50, about 50:50 to about 60:40, about 60:40 to about 70:30, about 70:30 to about 80:20, and about 80:20 to about 90:10.

Examples of polymers which form solid dispersions with EGCG include those that contain a cellulose functionality such as with HPMC-AS, HPMC-P, and cellulose acetate. Other polymer systems that form dispersions with EGCG include those that contain caprolactam functionalities such as polyvinyl caprolactam-polyvinyl acetate-polyethylene glycol graft co-polymer, one embodiment of which is Soluplus®.

The structure of Soluplus® is

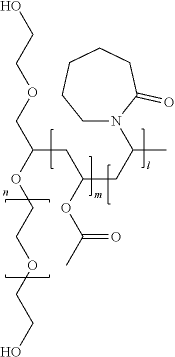

Where l, m, and n are values such that the average molecular weight, as determined by gel permeation chromatography is in the range of 90,000-140,000 g/mol.

The solid dispersions of EGCG and one or more polymers contain varying amounts of polymer. These weights range from about 9% to about 91% EGCG with the balance being polymer. All weights and weight ranges are reported as inputs prior to forming a solid dispersion.

In many embodiments of the invention, solid dispersions comprising amorphous EGCG and HPMC-AS are provided. In many of these embodiments, the solid dispersion has a glass transition temperature of less than about 80° C. including at about 72° C. In these and other embodiments, the solid dispersions exhibit an x-ray amorphous powder diffraction pattern, which remains after eleven days under accelerated stress conditions of 40° C. and 75% relative humidity. The term “x-ray amorphous” means that a powder diffraction pattern reveals one or more amorphous halos and does not contain sharp crystalline peaks. In some embodiments, the mass of EGCG and the mass of HPMC-AS is approximately the same in the solid dispersion (˜1:1 or equal to 1:1). In other embodiments, the weight of EGCG to HPMC-AS ranges from about 50:50 to about 91:9.

In other embodiments of the invention, solid dispersions of amorphous EGCG and HMPC-P is provided. In many of these embodiments, the solid dispersions exhibit an x-ray amorphous powder diffraction pattern and an x-ray amorphous diffraction pattern is seen after eleven days under accelerated stress conditions of 40° C. and 75% relative humidity. In some embodiments, the mass of EGCG and the mass of HPMC-P is approximately the same in the solid dispersion (˜1:1 or equal to 1:1). In other embodiments, the weight of EGCG to HPMC-P ranges from about 50:50 to about 91:9.

In other embodiments of the invention, solid dispersions of EGCG and cellulose acetate are provided. In many of these embodiments, the solid dispersions exhibit an x-ray amorphous powder diffraction pattern and an x-ray amorphous diffraction pattern is seen after eleven days under accelerated stress conditions of 40° C. and 75% relative humidity. In some embodiments, the mass of EGCG and the mass of cellulose acetate is approximately the same in the solid dispersion (˜1:1 or equal to 1:1). In other embodiments, the weight of EGCG to cellulose acetate ranges from about 50:50 to about 91:9.

In many embodiments of the invention, solid dispersions comprising amorphous EGCG and polyvinyl caprolactam-polyvinyl acetate-polyethylene glycol graft co-polymer is provided. In many of these embodiments, the solid dispersions have a glass transition temperature of less than about 150° C. including at about 143° C. In these and other embodiments, the solid dispersion exhibits an x-ray amorphous powder diffraction pattern and an x-ray amorphous diffraction pattern is seen after eleven days under accelerated stress conditions of 40° C. and 75% relative humidity. One tradename of such a polymer is Soluplus®. In these and other embodiments, the amount of EGCG released within 20 minutes in a pH 7.4 medium containing PBS is less than 60% and in some embodiments is about 50%. The amount of EGCG released in many of the solid dispersions of the invention is less than that found in corresponding crystalline EGCG. In some embodiments, the mass of EGCG and the mass of polyvinyl caprolactam-polyvinyl acetate-polyethylene glycol graft co-polymer is approximately the same in the solid dispersion (˜1:1 or equal to 1:1). In many embodiments, the ratio of polyvinyl caprolactam-polyvinyl acetate-polyethylene glycol graft co-polymer to EGCG is between about 10:1 to about 1:10 including between about 91:1 to about 9:91. In certain of the embodiments, polyvinyl caprolactam-polyvinyl acetate-polyethylene glycol graft co-polymer is Soluplus®.

It was previously reported that EGCG tends to bind with polyvinyl pyrrolidone and polyvinyl polypyrrolidone, resulting in colloids and aggregates (Patel et al., 2011). These colloids are typically undesirable, but may find applications of special formulations in food or drugs, whereas they could also raise difficulties in formulation and reduce efficacy of polyphenols (Patel et al., 2011). This may explain why precipitates were observed upon mixing EGCG solutions with polymer solutions of PVP, PVA, PEG, and PVAC. Furthermore, such binding may occur, though likely weaker, between EGCG and the other five polymers of HPMC-AS, HPMC-P, Soluplus®, cellulose acetate, and Gelucire® 50/13 listed in Table 5 since during mixing of solutions, samples can be cloudy but clear again upon addition of more solvent and vortexing. One intrinsic difference between amorphous dispersions and colloidal complexes is the molecular-level mixing of the drug and the polymer. With colloidal complexes, cloudy solutions with precipitates form. To reduce the chance of colloid formation, a slow and stepwise addition approach was employed for the dispersions, along with adding additional fresh solvents and vortexing for the purpose of acquiring clear solutions.

In many embodiments, clear solutions containing EGCG and polymer were lyophilized to yield solids. The lyophilized materials were often fluffy and lightweight. For example, the Soluplus® 50:50 dispersion in one embodiment showed a white color while all the other dispersions and the EGCG displayed some yellowness. Solids were then characterized by XRPD, and the results are included in Table 5. Among them, Gelucire® 50/13 as prepared was not a suitable candidate to generate amorphous dispersion since the lyophilized solids displayed a disordered x-ray diffraction pattern with characteristic peaks consistent with Gelucire® 50/13. As a result, four polymers, i.e., HPMC-AS, HPMC-P, Soluplus®, and cellulose acetate, were selected as candidate excipients to generate amorphous dispersions with EGCG for further study.

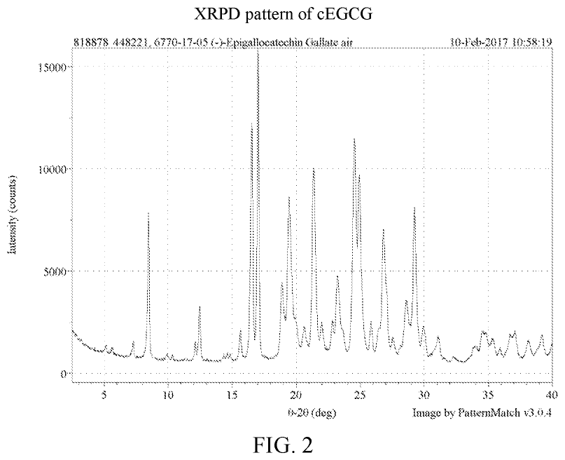

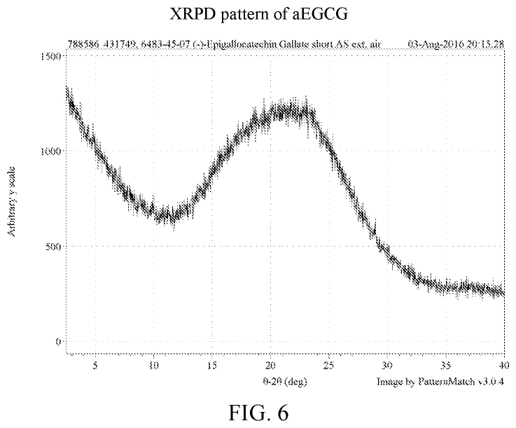

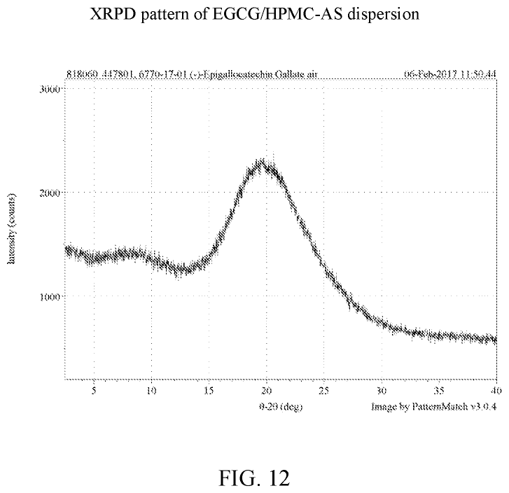

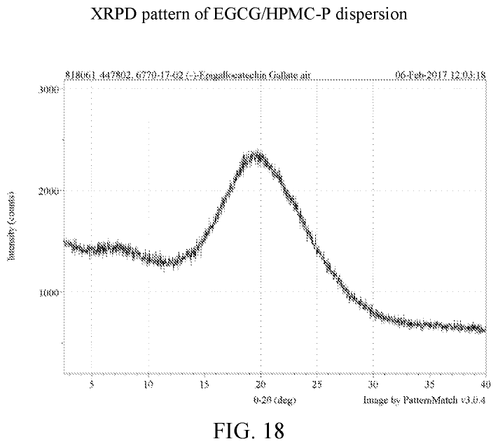

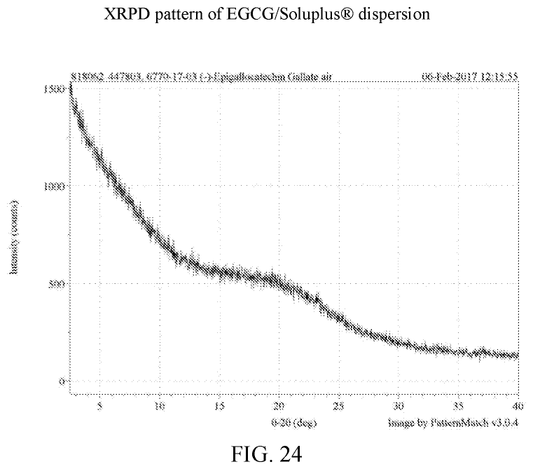

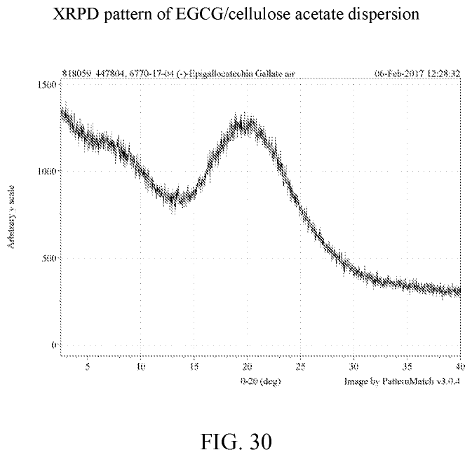

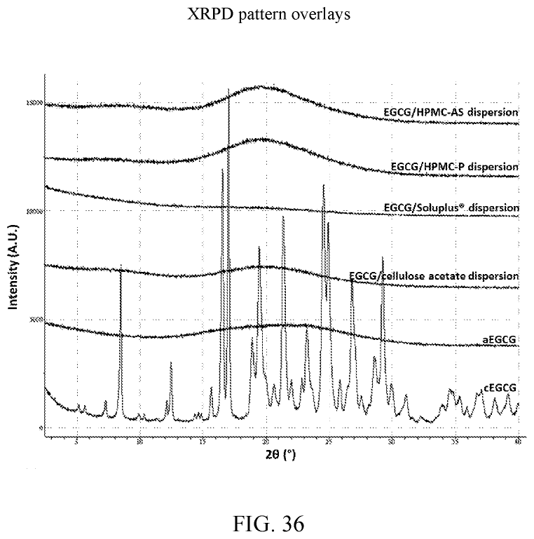

EGCG amorphous solid dispersions generated by lyophilization were characterized by XRPD and the data compared to the lyophilized EGCG and the starting material of EGCG, as shown in FIG. 36. Based on the results, the four dispersions and the lyophilized EGCG show clear x-ray amorphous patterns while the EGCG isolated from Teavigo™ is a crystalline material. The individual x-ray powder diffractions of cEGCG, aEGCG, EGCG/HPMC-AS, EGCG/HPMC-P, EGCG/Soluplus®, and EGCG/cellulose acetate can be found at FIGS. 2, 6, 12, 18, 24, and 30 respectively.

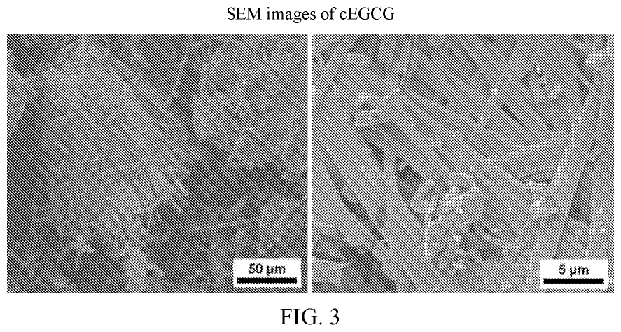

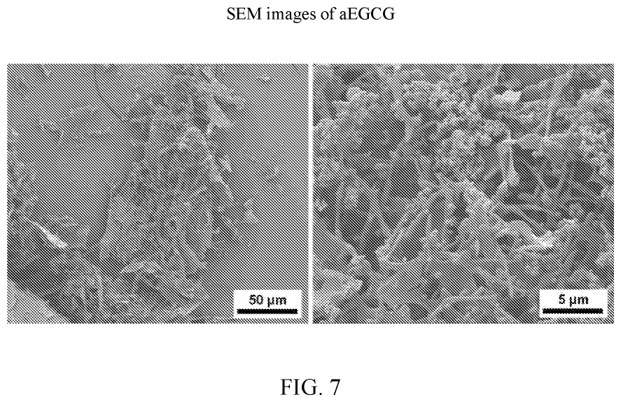

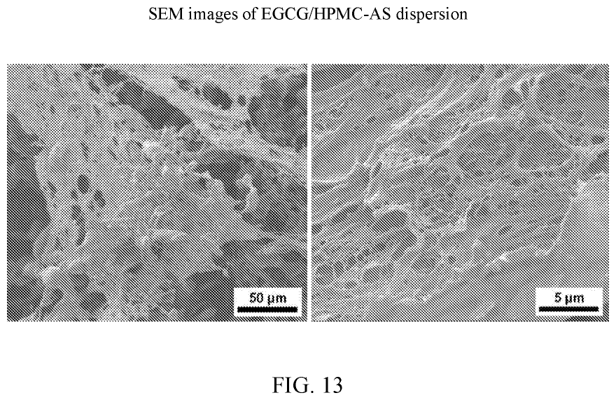

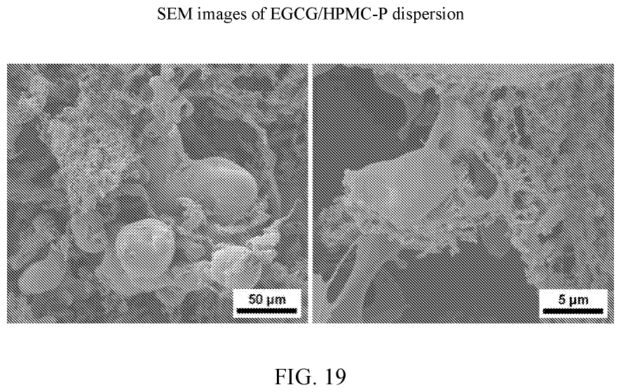

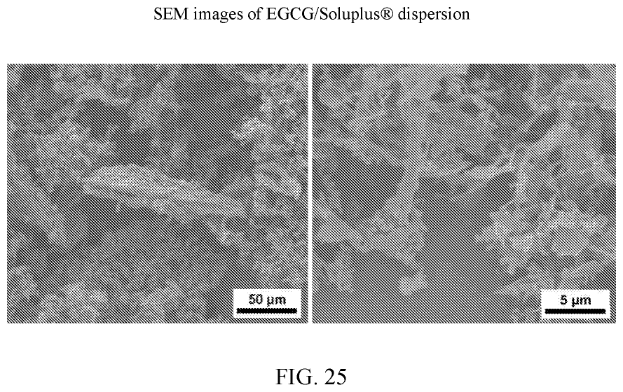

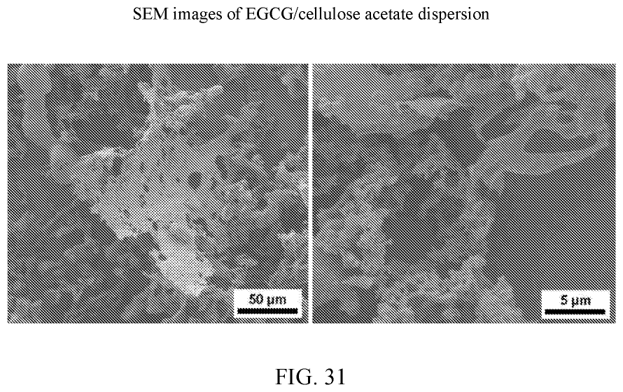

SEM images of cEGCG, aEGCG, and the four dispersions are provided in FIGS. 3, 7, 13, 19, 25, and 31 respectively. The cEGCG and aEGCG SEM images exhibit disparate morphologies. The cEGCG micrograph contains long thin flat laths while the aEGCG micrograph has continuous structure consisting of round-ended fibers and small spheres with a broad distribution of particle sizes down to tens of nm. The four dispersions all show continuous structures with different morphologies as well. For the one with HPMC-AS (FIG. 13), EGCG particles appear intimately interconnected with the polymer forming a porous layered network. The dispersions with HPMC-P (FIG. 19) and cellulose acetate (FIG. 31) exhibit relatively separated phases of EGCG and polymer. The Soluplus® dispersion shows a higher level of homogeneity and with no apparent separation between EGCG and polymer (FIG. 25).

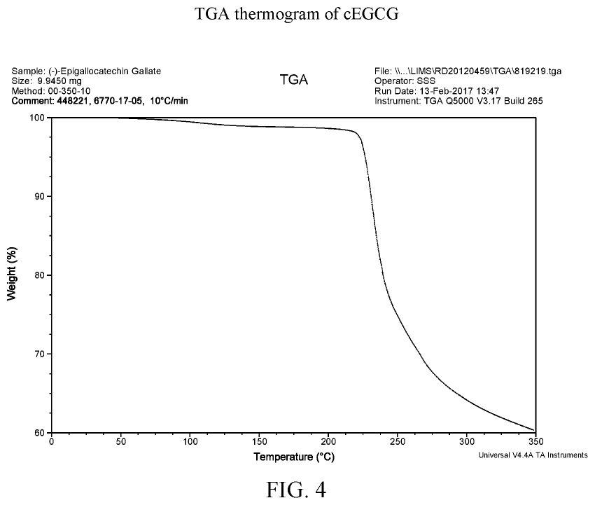

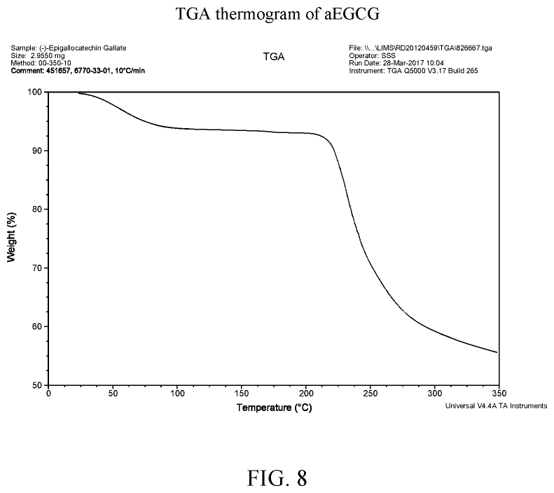

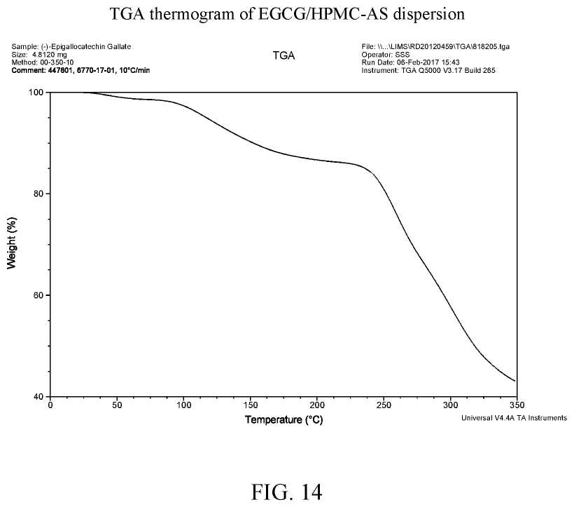

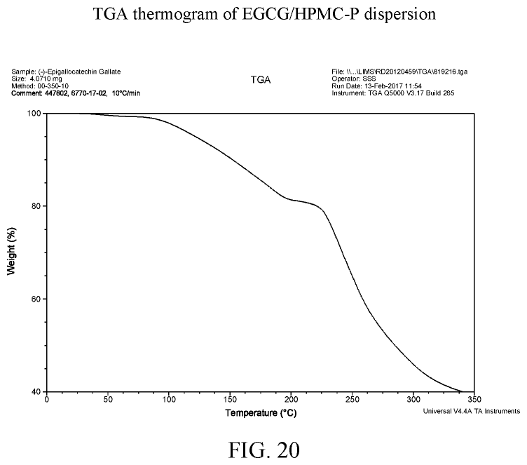

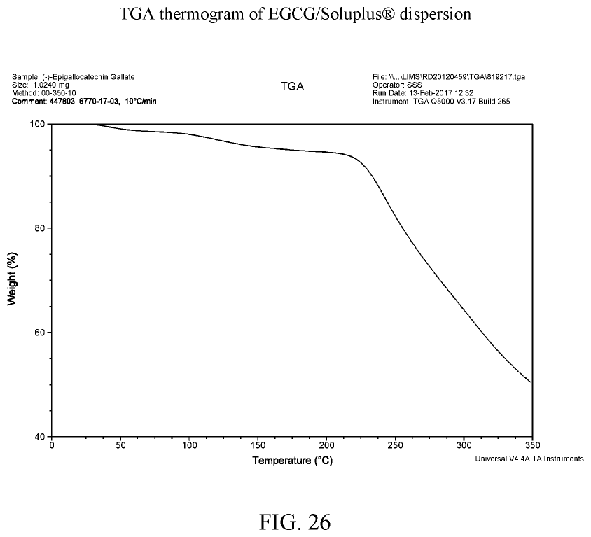

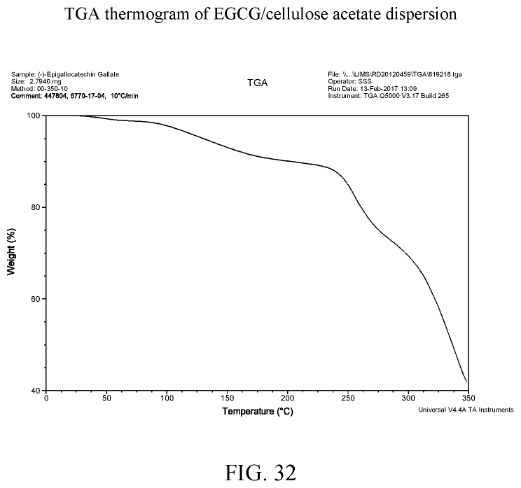

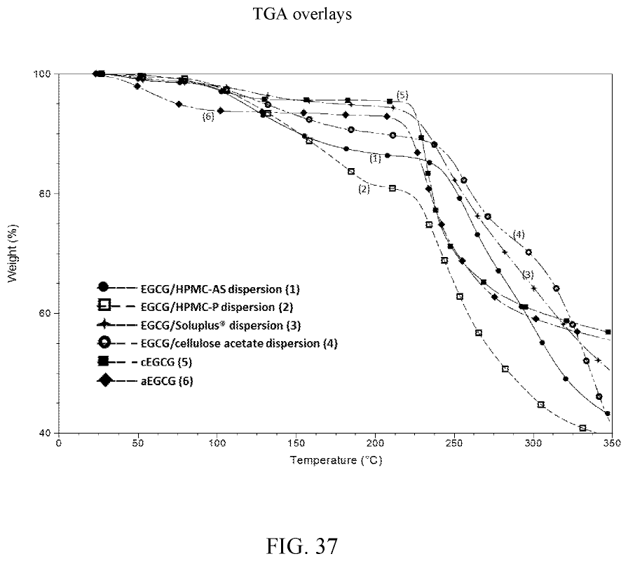

FIG. 37 provides the TGA data of the four vacuum-dried disperions and both aEGCG and cEGCG. Certain amounts of volatiles are observed in all of samples. For EGCG, the water is the volatile while for the dispersions, volatiles could be water, dioxane or a mixture of both. The amount of volatiles are estimated and accounted for when calculating the EGCG equivalency for dissolution testing. Starting from approximately 220° C., all materials show apparent decomposition. Individual TGA curves are shown at FIGS. 4, 8, 14, 20, 26, and 32 for cEGCG, aEGCG, EGCG/HPMC-AS, EGCG/HMPC-P, EGCG/Soluplus®, and EGCG/cellulose acetate respectively.

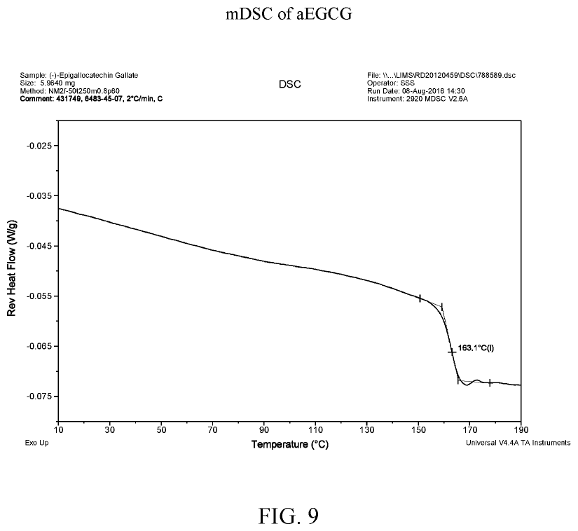

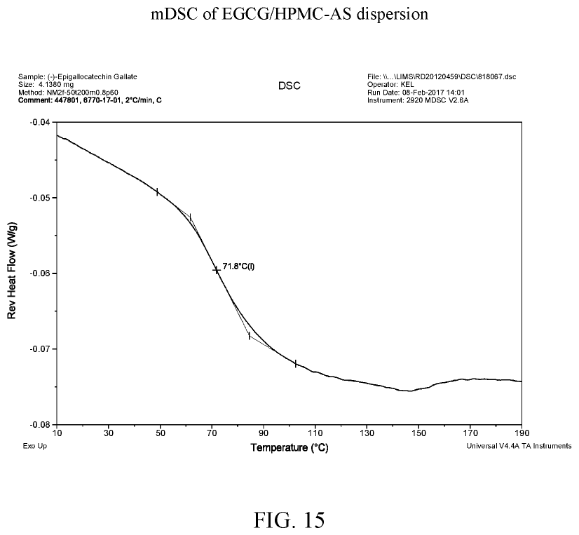

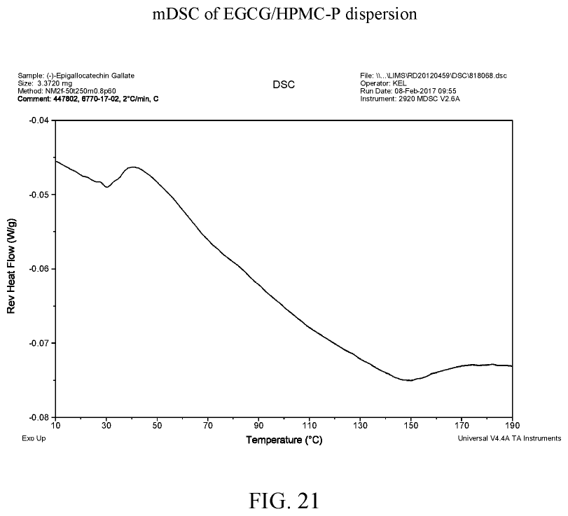

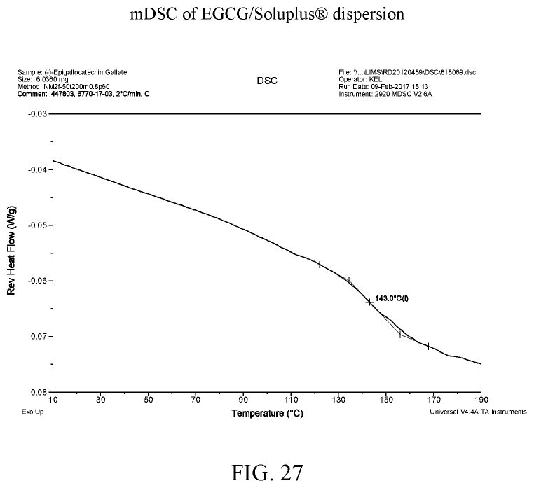

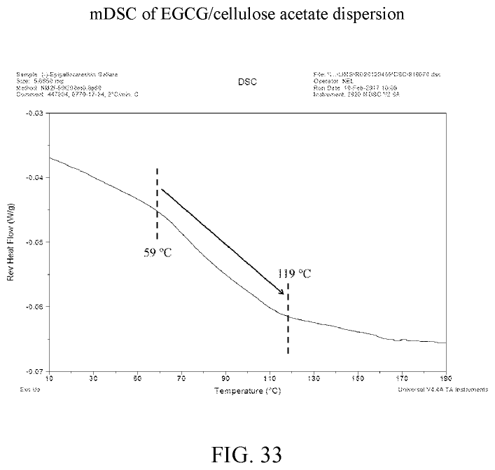

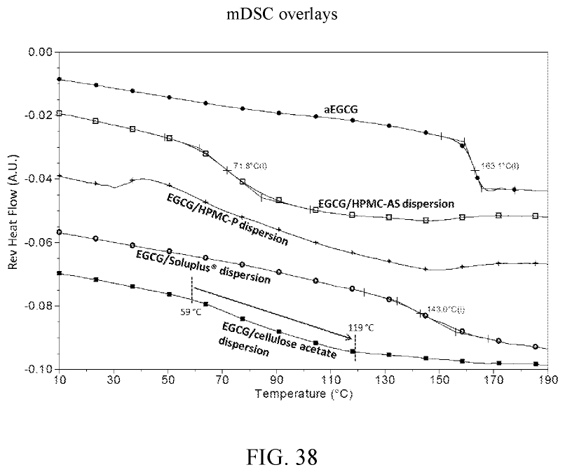

FIG. 38 shows the mDSC data for aEGCG and the four dispersions. Glass transition temperatures are observed at about 163° C., 72° C., and 143° C. for aEGCG, the dispersions of EGCG/HPMC-AS and EGCG/Soluplus® respectively (see FIGS. 9, 15, and 27 respectively). No clear glass transition is observed for the EGCG/HPMC-P dispersion (see FIG. 21). For the EGCG/cellulose acetate dispersion, the glass transition spanned a large range of 59-119° C. (see FIG. 33). The presence of one (miscible) or two (phase separation) glass transition is often used to evaluate drug-polymer miscibility (Baird and Taylor, 2012). For example, the EGCG/HPMC-AS and EGCG/Soluplus® dispersions display only one apparent T_{g}, suggesting that EGCG and the polymers are likely miscible in the materials.

The physical stability of aEGCG and the four dispersions were evaluated at different conditions including a typical stress condition of 40° C./75% RH for 11 days, and in media of SGF and SIF for 24 h. Table 1 summarizes the visual observations on the materials after they have been stressed at 40° C./75% RH for 11 days. The aEGCG became a hard material after stress, while all the dispersions were particles or soft aggregates.

TABLE 1
Observation of the materials after
40° C./75% RH stress for 11 days

| Material | Visual observation after stress |
|---|---|
| aEGCG | pink hard material, agg. |
| EGCG/HPMC-AS | pink agg. and particles |
| EGCG/HPMC-P | pink agg. and particles |
| EGCG/Soluplus ® | white free flowing agg. and particles |
| EGCG/cellulose acetate | dark pink agg. and particles |

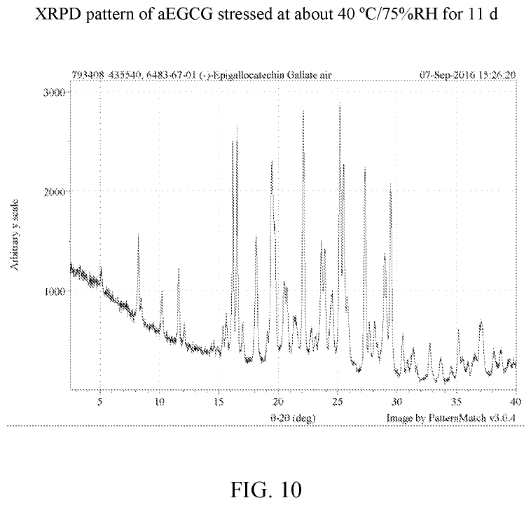

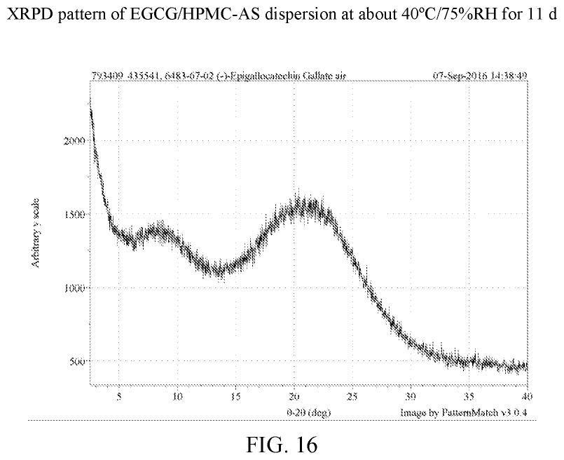

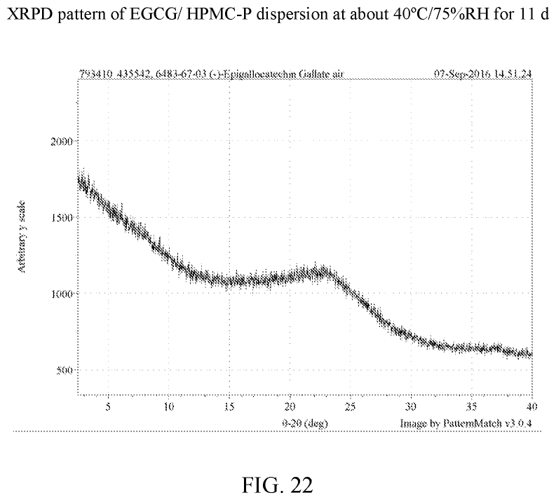

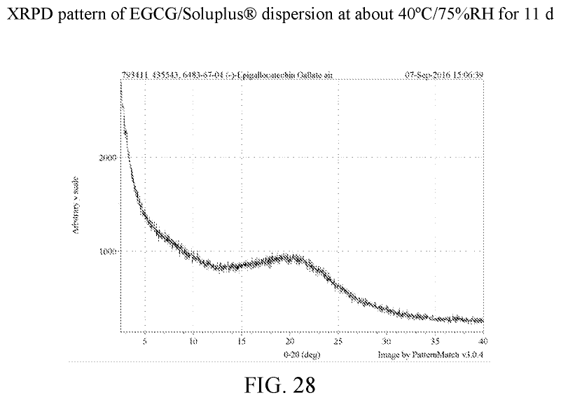

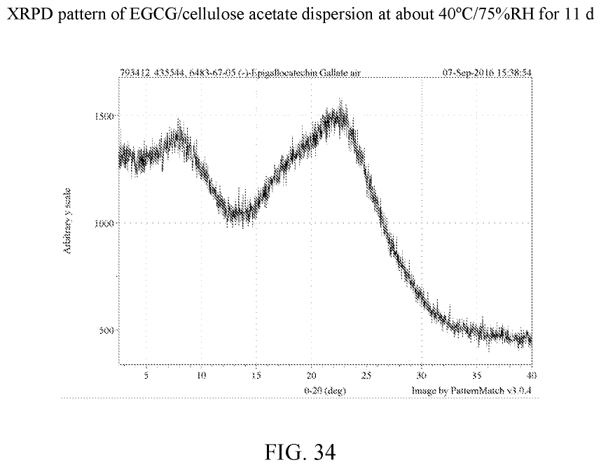

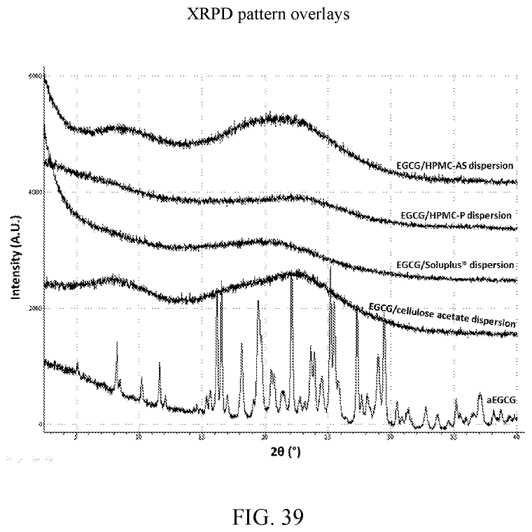

The post-stress samples were characterized by XRPD and the results are presented in FIG. 39. The aEGCG turned into a highly crystalline material while the four dispersions remained x-ray amorphous, suggesting the dispersions are physically stable at this stress condition. In addition, the Soluplus® dispersion retained its white color after stress while all the others presented different levels of color changes. It has been well established that the color change of EGCG directly correlated with its chemical instability, i.e., degradation and oxidation (Li et al., 2013; Sang et al., 2005). This difference in appearance after stress likely indicates that the Soluplus® dispersion has a higher chemical stability than other dispersions and aEGCG. This improved physicochemical stability may have significant impacts on the whole cycle of storage, transportation, and processing of EGCG. Individual diffraction patterns for such stressed conditions can be found at FIGS. 10, 16, 22, 28, and 34 for aEGCG, EGCG/HPMC-AS, EGCG/HPMC-P, EGCG/Soluplus®, and EGCG/cellulose acetate respectively.

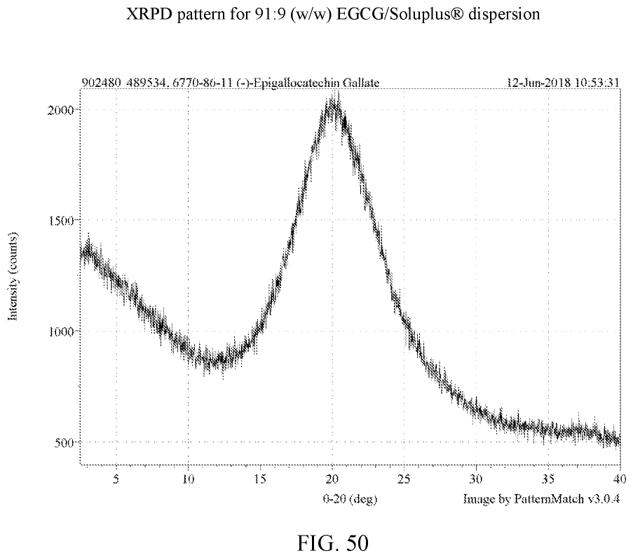

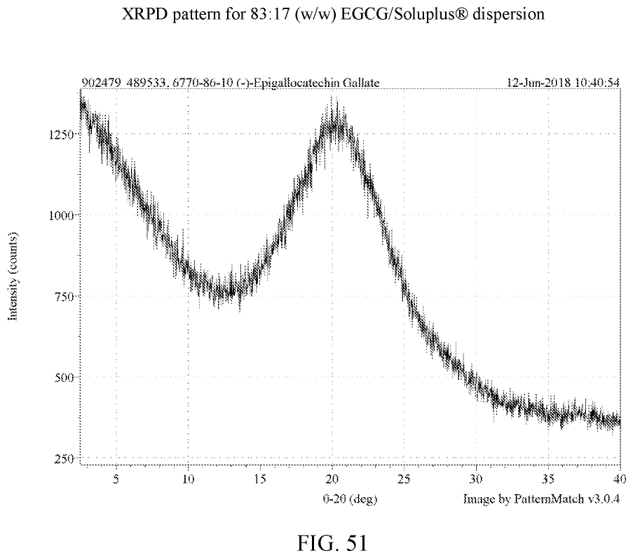

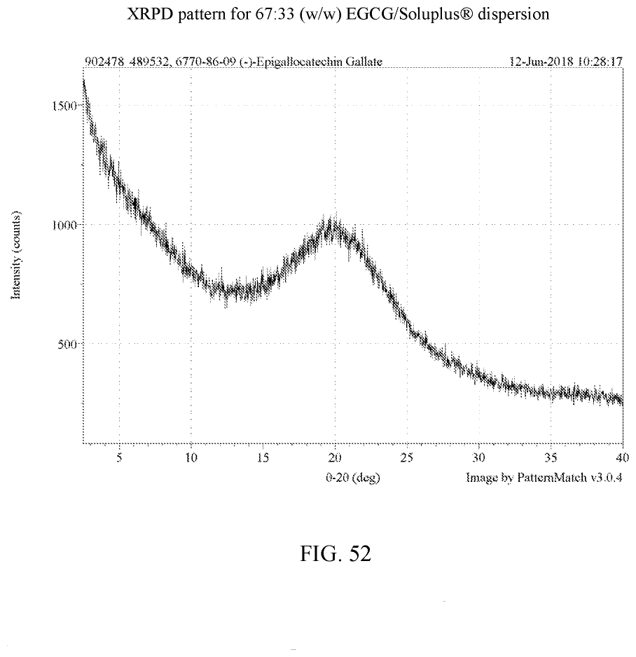

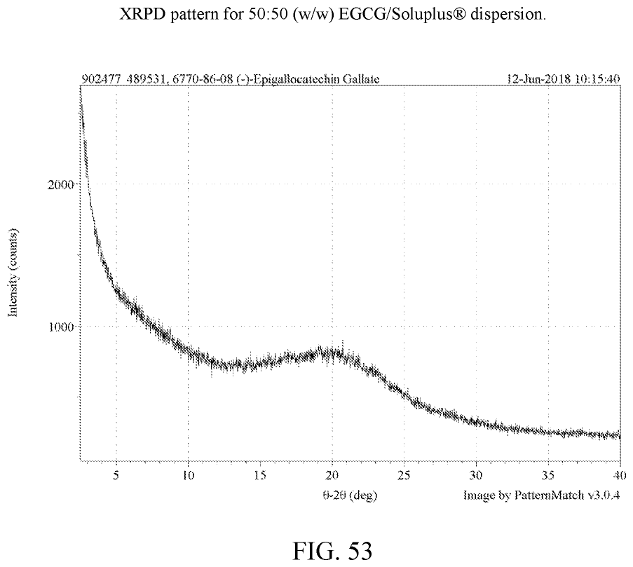

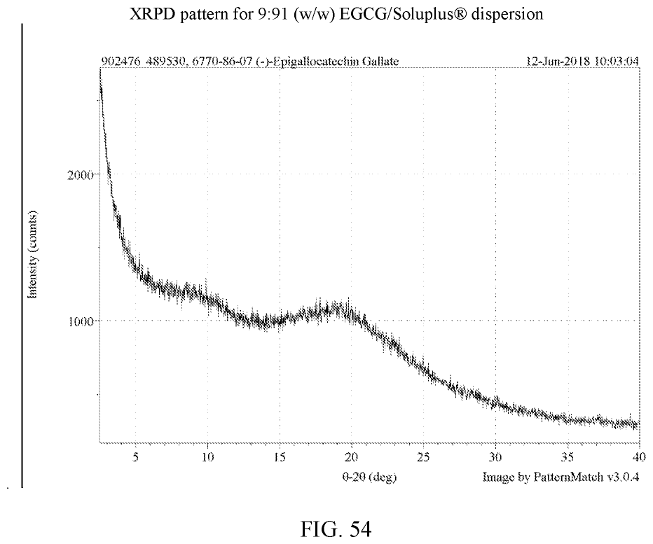

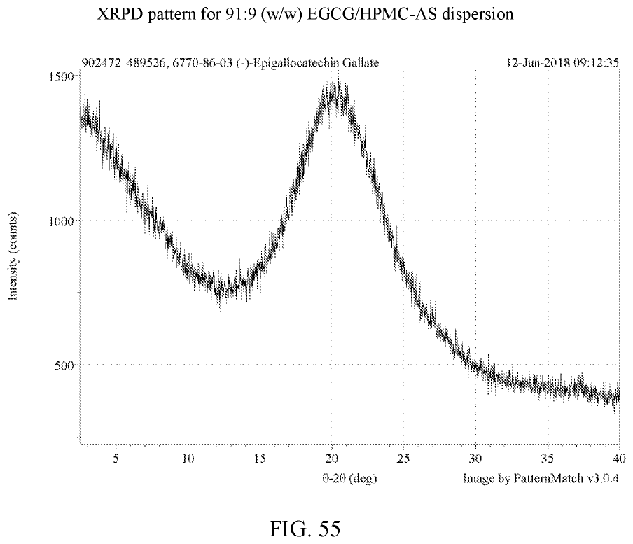

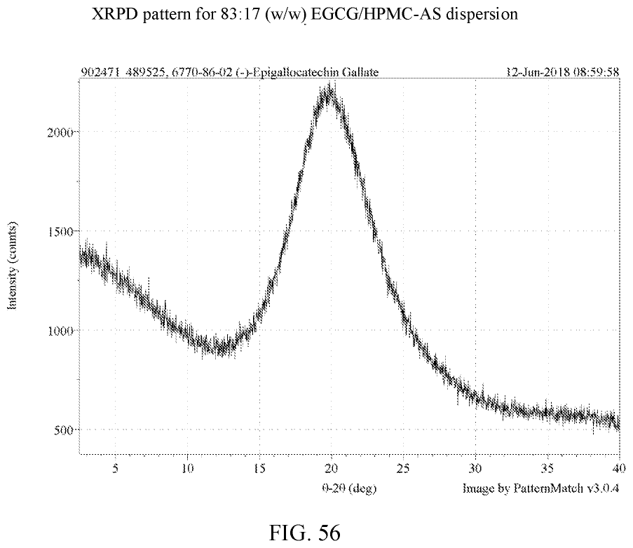

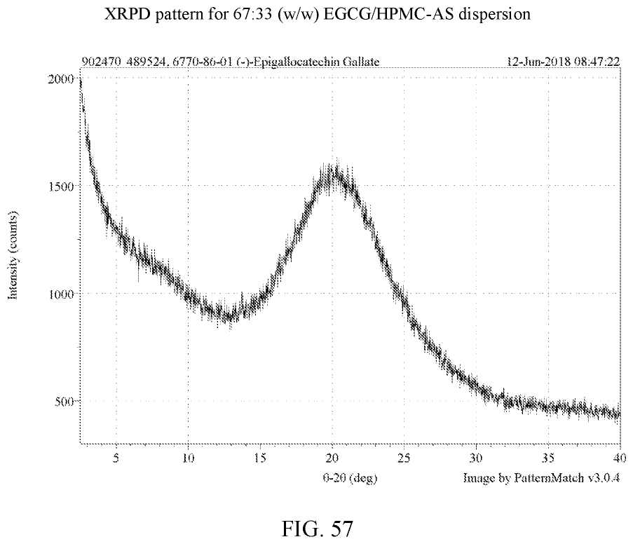

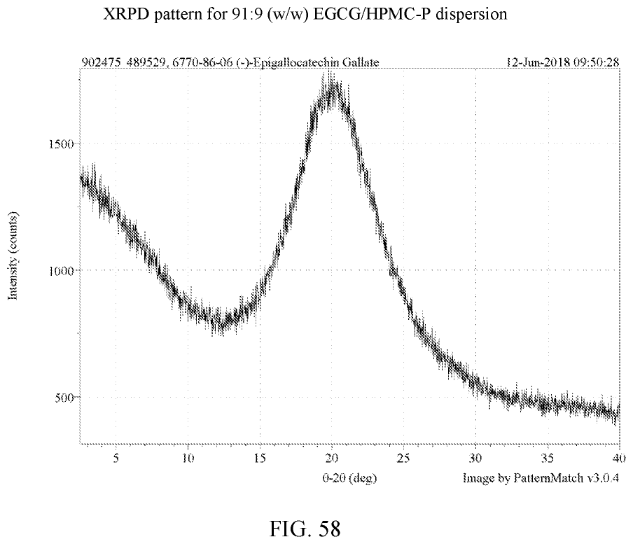

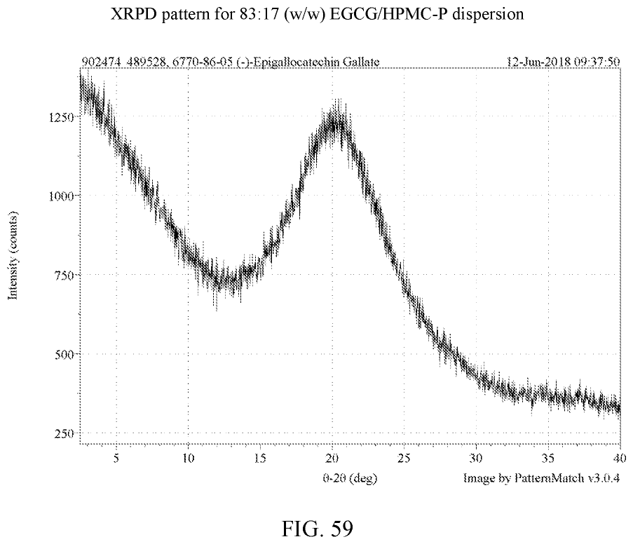

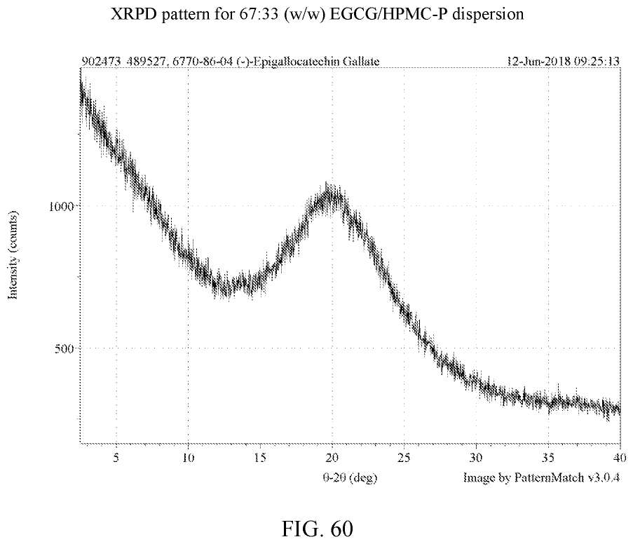

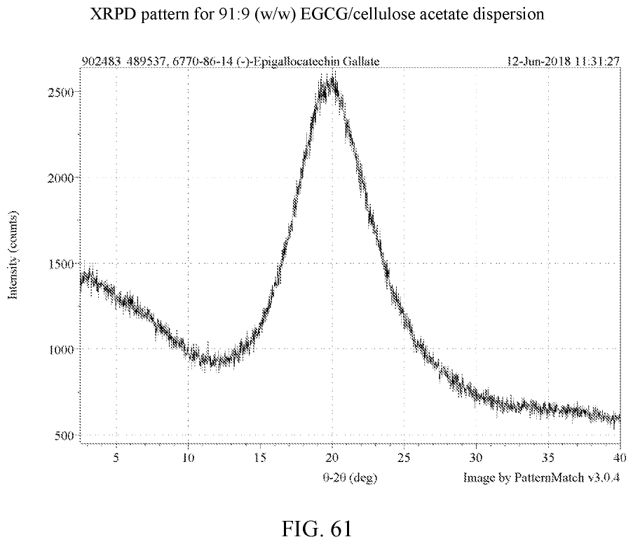

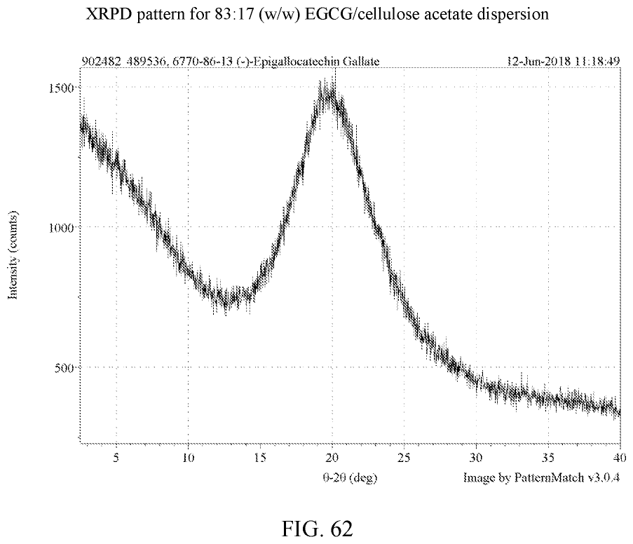

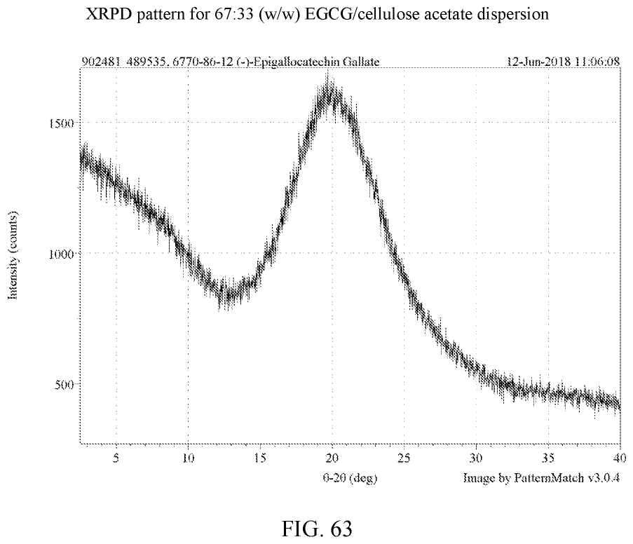

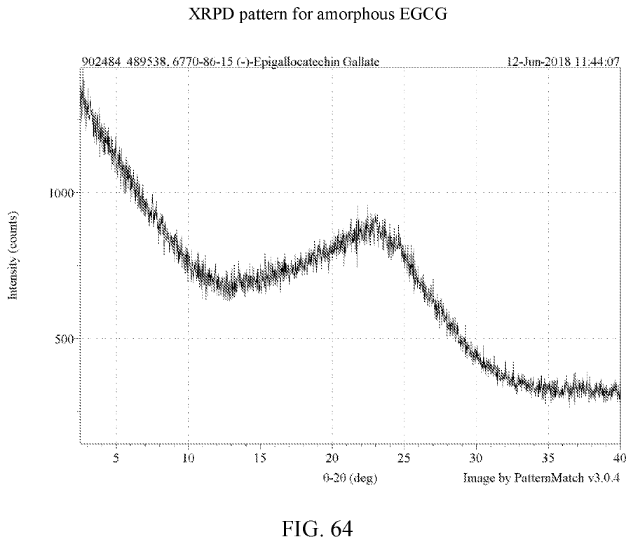

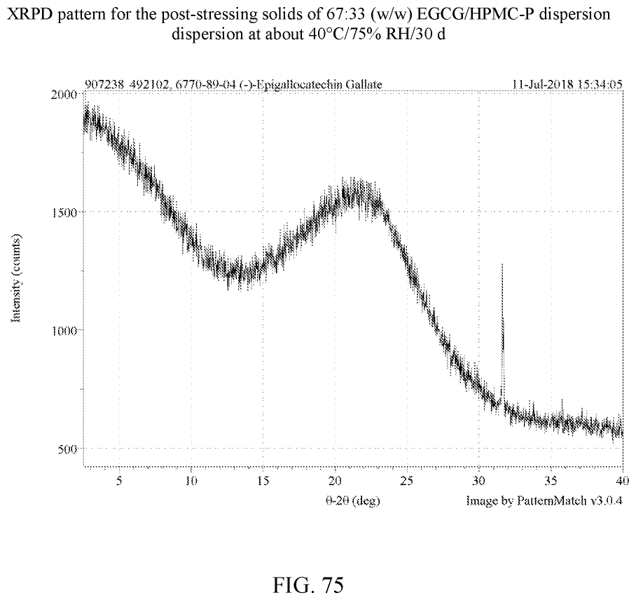

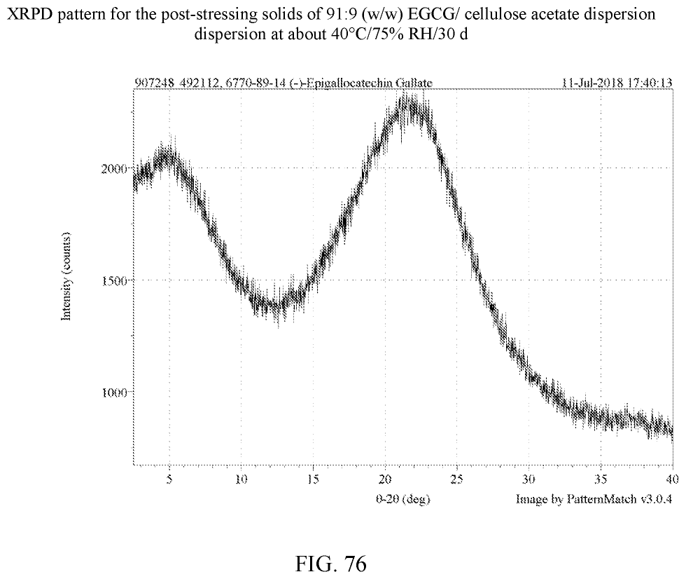

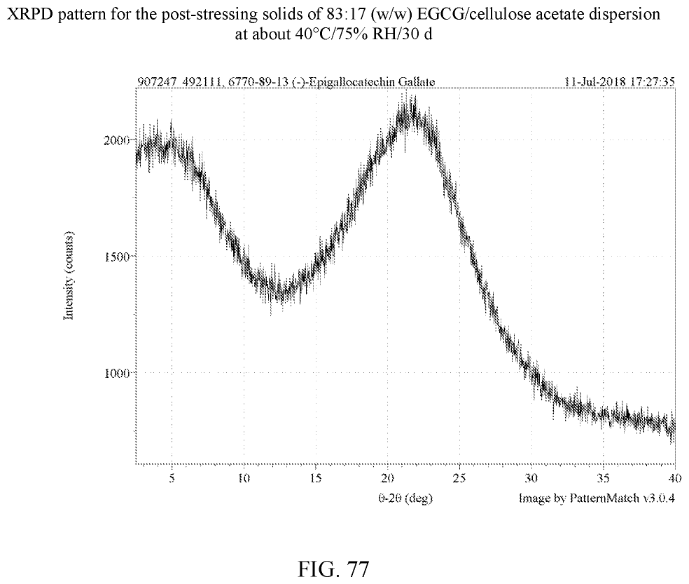

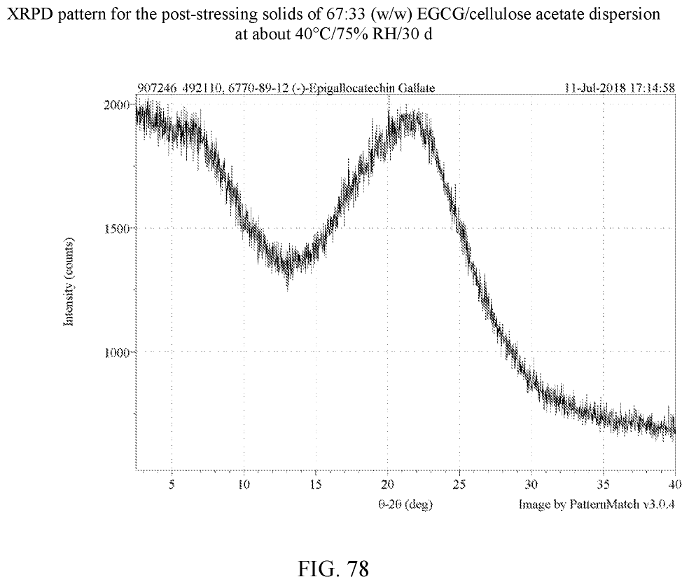

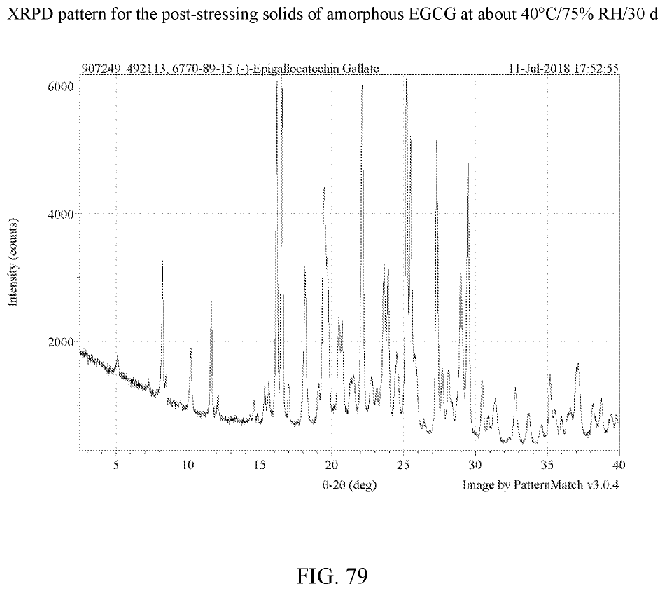

For the four polymers identified, further stress tests were carried out on dispersions with varying weight ratios of EGCG to polymer as set forth in Example 8. In these experiments, stressing was done for 30 days at 75% relative humidity at 40° C. for 30 days. A total of 14 dispersions were made in H₂O and dioxane as shown in Table 9. FIGS. 50-63 are the XRPD patterns of the prepared dispersions prior to stressing. FIGS. 75-78 are the XRPD patterns after stressing and these too show the samples to amorphous by XRPD. FIGS. 82, 83, and 85 show a peak indicative of NaCl, which is used in the salt both of the humidity chamber storing the samples. Thus, such a peak is not indicative of any crystalline EGCG in the corresponding dispersions. As a control, an EGCG was prepared (FIG. 64) and similarly stressed and it showed conversion to EGCG as seen in FIG. 79. Based on the XRPD results, the post-stressing dispersions were x-ray amorphous (other than the NaCl peaks).

Table 10 shows color changes after stressing. A color change may be indicative of chemical degradation to some degree. With regards to the tested dispersions, all the dispersions prepared had a very light pinkish/off white hue. After stressing, the 50:50 EGCG:Soluplus® dispersion remained unchanged and the 9:91 EGCG:Soluplus® dispersion had a slight change to light yellow. The other Soluplus® dispersions changed to a dark red whereas the dispersions with other polymers changed to a very dark red which was almost black in hue. The color change suggested that the Soluplus® dispersions had the least degradation of the dispersions tested. The non-dispersed amorphous EGCG converted to crystalline EGCG.

For stress testing in media, solids of aEGCG and the dispersions were suspended in SGFs and SIFs (see Example 6), and the suspensions were observed under polarized light microscope from time to time to look for the evidence of birefringence and extinction (B/E) of the materials, which is the indication of the occurrence of crystallization in a material. The observations are summarized in Table 2. Amorphous EGCG crystallized quickly, within 15 minutes in both SGF and SIF, while no occurrence of crystallization was noticed for the four dispersions in both media for at least 24 h, suggesting improved physical stability in the GI environment of the dispersions compared to aEGCG.

TABLE 2
Observations under PLM for the dispersions
and aEGCG in SGF and SIF media

| | | |
|---|---|---|
| Material | in SGF | in SIF |

| | |
|---|---|
| EGCG | B/E observed in 15 min |
| EGCG/HPMC-AS | no B/E observed in 24 h |

EGCG/HPMC-P
EGCG/Soluplus ®
EGCG/cellulose acetate

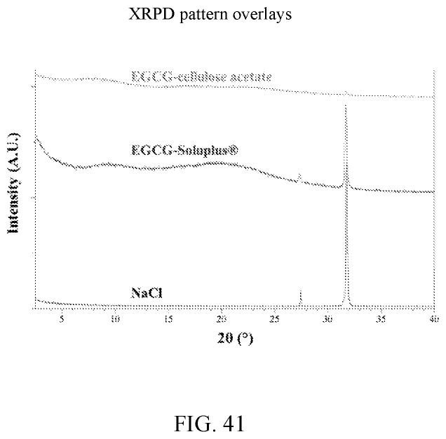

The dissolution tests for the EGCG and dispersions were carried out in Example 7 to determine the release profiles of EGCG in a GI tract model. During tests, cEGCG, aEGCG, EGCG dispersions prepared with HPMC-AS or HPMC-P were all dissolved without visible solids, while residual solids were present for dispersions of EGCG/Soluplus® and EGCG/cellulose acetate after 24 hours. The solids were isolated, dried under N₂ purge and then analyzed by XRPD. The XRPD patterns of the post-dissolution solids of EGCG/Soluplus® and EGCG/cellulose acetate dispersions, along with the pattern of NaCl are shown in FIG. 41. The peaks which appeared in the XRPD patterns of both solids are consistent with NaCl which came from the dissolution medium. No evidence of crystalline peaks consistent with crystalline EGCG is observed. In fact, based on the dissolution data, for both samples all EGCG (˜20 mg) was released into the medium within the period of dissolution tests. Therefore, the solids isolated after dissolution are un-dissolved polymers, Soluplus® or cellulose acetate, respectively.

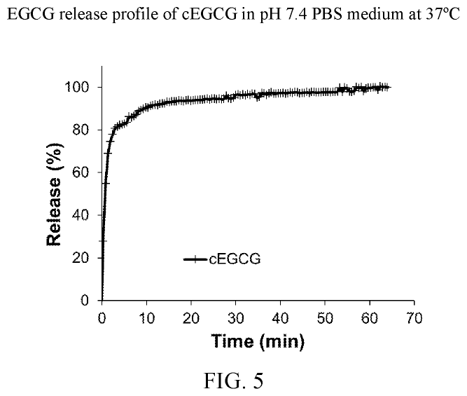

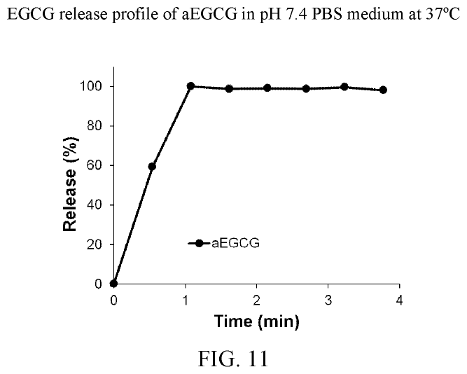

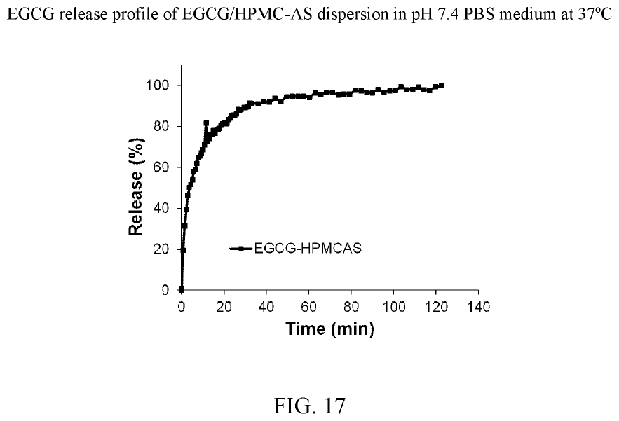

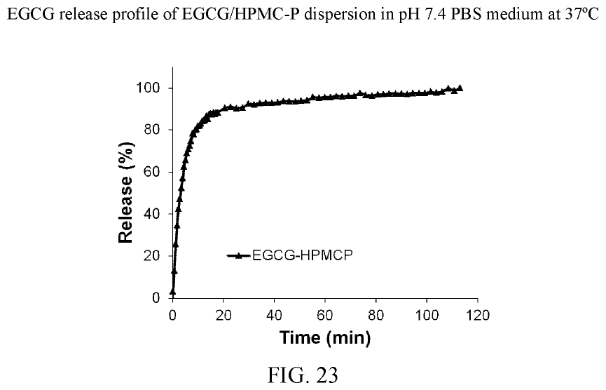

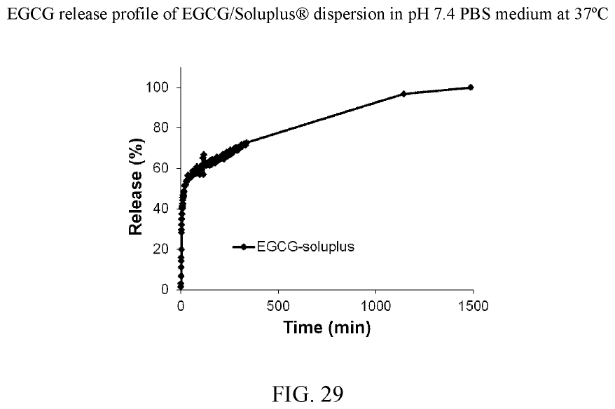

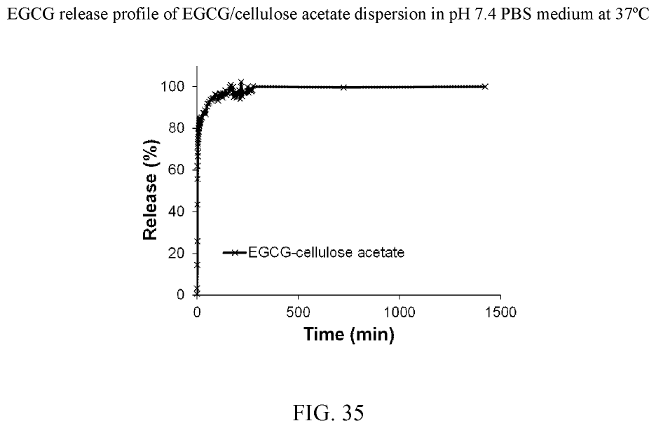

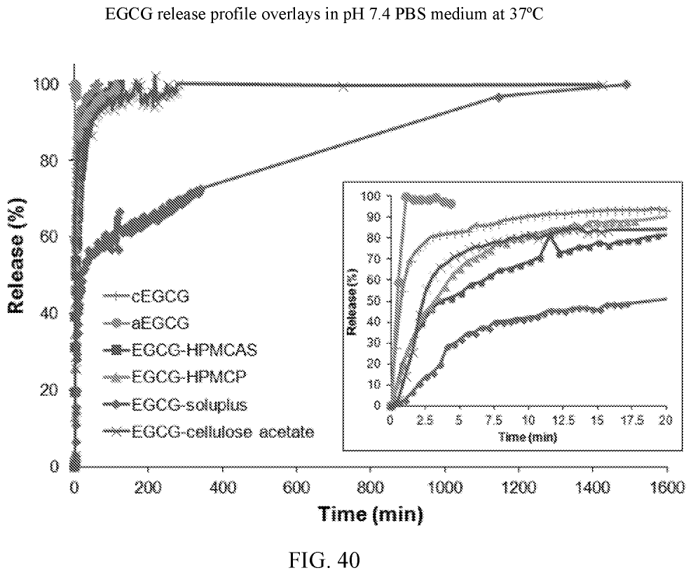

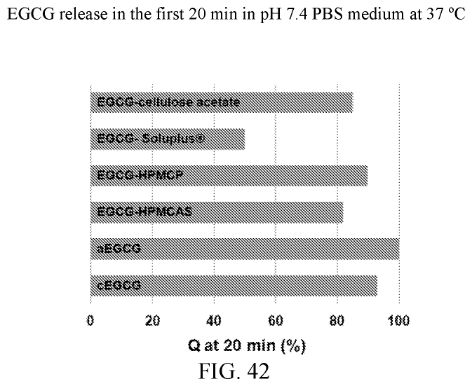

The release profiles for each material are presented in FIG. 40 and the data in the first 20 minutes are presented in the inset. During this 20 minutes, the releases of EGCG were approximately 93% for cEGCG, 100% for aEGCG, 82% for EGCG/HPMC-AS, 90% for EGCG/HPMC-P, 50% for EGCG/Soluplus®, and 85% for EGCG/cellulose acetate, respectively, as shown in FIG. 42. The individual release profiles are found at FIGS. 5, 11, 17, 23, 29, and 35 for cEGCG, aEGCG, EGCG/HPMC-AS, EGCG/HPMC-P, EGCG/Soluplus®, and EGCG/cellulose acetate respectively.

As expected, aEGCG exhibits an immediate dissolution, at a much higher rate than all the other materials. The three dispersions with HPMC-AS, HPMC-P, and cellulose acetates show slower release compared to cEGCG. For dispersions with HPMC-AS and HPMC-P, since the polymers are quickly dissolved in the medium, they are no longer able to prevent EGCG molecules from releasing. Under this situation, the interactions between polymers and EGCG do not significantly prolong the release of EGCG in the intestine (high pH). Based on the release profiles for the first 20 minutes (FIG. 40), the dispersions of HPMC-P and cellulose acetate have the faster dissolutions than the other two dispersions. For the Soluplus® dispersion, a distinct release profile is observed compared to the other materials. Only half of the EGCG is released in the first 20 minutes and the dissolution continues up to approximately 24 hours.

Without being bound by theory, it is believed that the micelle-forming ability of polyvinyl caprolactam-polyvinyl acetate-polyethylene glycol graft co-polymer or a Soluplus® based polymer enhances the performance of the resulting solid dispersion which provides for the sustained release of EGCG. In support of this theory, the release kinetics of the EGCG/Soluplus® dispersion was modeled and compared with the other three dispersions as well as aEGCG and cEGCG.

As an initial proposition, the dispersions, aEGCG and cEGCG were modeled with respect to pseudo-second order models according to equation 1 where t is time, Q is cumulative release, and Qₑ the final release and, and k₂ the rate constant of pseudo-second order kinetics.

$$\frac{t}{Q} = \frac{1}{{k}_{2} {Q}_{e}^{2}} + \frac{t}{{Q}_{e}} \qquad Eq . ( 1 )$$

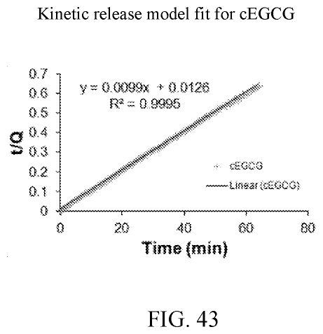

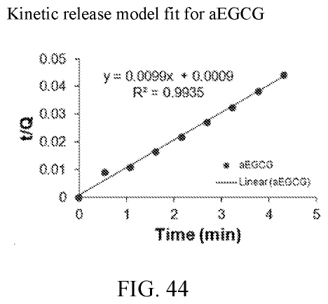

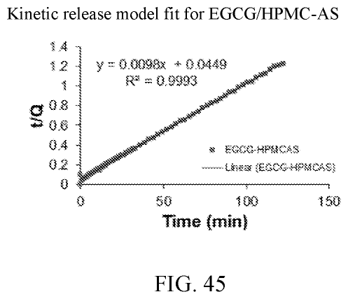

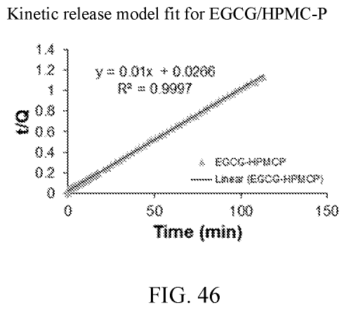

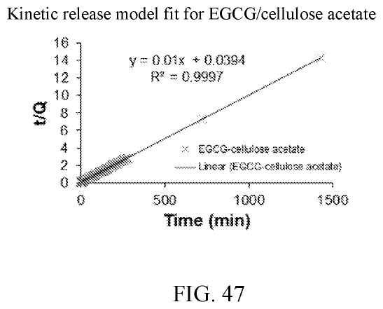

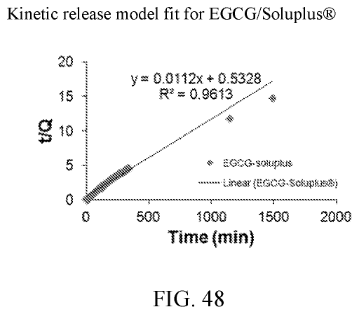

Pseudo-second order kinetics is often used to model the release and/or adsorption of materials in aqueous media. In this case, all of the dispersions and aEGCG and cEGCG fit this model as evidenced by very high correlation coefficients (FIGS. 43-47). The outlier was EGCG/Soluplus® which fit the model poorly with a correlation coefficient of only 0.9613 showing substantial nonlinear behavior with this model (FIG. 48).

Soluplus®, as a polyvinyl caprolactam-polyvinyl acetate-polyethylene glycol graft copolymer, has an amphiphilic structure with a high lipophilicity, and possesses a very low critical micelle concentration (7.6 mg/L) (Reintjes, 2011). Soluplus® has a bifunctional character, which can be used as a matrix polymer for solid solutions and also an active solubilizer through micelle formation in water (Shamma and Basha, 2013). A number of reports have demonstrated the use of self-assembled polymer micelles based on amphiphilic block copolymers as vehicles to improve delivery and bioavailability (Bontha et al., 2006; Huynh et al., 2009; Kim et al., 2009; Liu et al., 2006; Siddiqui et al., 2009).

During the dispersion generation with EGCG, Soluplus® reached a concentration of 7353 mg/L in water-dioxane solution prior to lyophilization. This concentration is much higher than its critical micelle concentration, and therefore Soluplus® micelles likely formed in the solution with EGCG molecules inside the micelles. Micelle formation would result in a tight wrapping of the EGCG molecules and/or the high miscibility between Soluplus® and EGCG, which may explain why they yield lyophilized solids with white color while the other dispersions show yellowness originating from the color of EGCG (see Table 1). In the dissolution test, it is hypothesized that the tightly wrapped EGCG is released after the dissolution or at least partial dissolution of Soluplus®, which could form micelles again in the dissolution medium and further delay the release of EGCG.

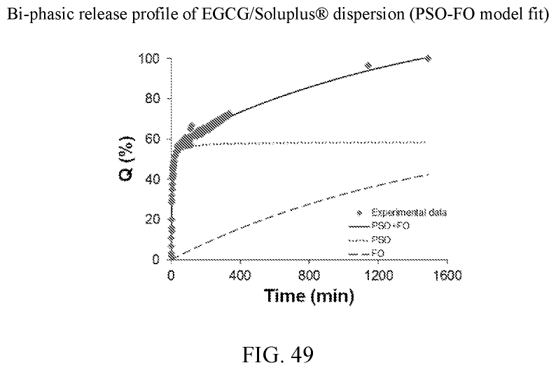

With the strong tendency to form micelles, the interaction between EGCG and water is mitigated. Thus, while the hydroxyls of EGCG render an adequate binding with the hydrophilic chains in Soluplus®, the high lipophilicity of Soluplus® lowers the wettability of EGCG. Indeed, these features are consistent with a bi-phasic release of EGCG in a Soluplus® dispersion (see FIG. 49). In the first portion of the release, EGCG molecules that are not fully enveloped by the dispersive protection of the micelles of EGCG are released in the same manner as the other dispersions whereas EGCG that is within the micelles are released at a slower rate due to the water-inhibition properties of the micelles.

This biphasic model that combines pseudo-second-order and first-order kinetics is used to describe the release profile of the EGCG/Soluplus® dispersion as set forth in equation 2:

$${Q}_{t} = \frac{t}{1 / {k}_{2} {Q}_{e}^{2} + 1 / {Q}_{e}} + {Q}_{0} ( 1 - exp ( - {k}_{1} t ) ) \qquad EQ . ( 2 )$$

The first order portion of equation (2) is also a special case of the Weibull model:

$$Q = {Q}_{0} ( 1 - exp ( - \frac{{( t - T )}^{b}}{a} ) ) \qquad EQ . ( 3 )$$

Other polymers which exhibit micelle forming properties include amphiphilic polymers such as poly(ethylene oxide)-poly (propylene oxide)—poly(ethylene oxide) polymers. Other micelle-forming polymers include those set forth in Table 3.

TABLE 3
Micelle-Forming Polymers

| | | | | |
|---|---|---|---|---|
| PEO-PBLA | PEO-P(Lys) | PEO-P(Asp) | PEO-PE | PEO-PDLLA |
| poly(ethylene oxide)- | poly(ethylene | poly(ethylene | poly(ethylene | poly(ethylene |
| poly(J3-benzyl-L-aspartate) | oxide)-poly(L- | oxide)- | oxide)- | oxide)-poly(DL- |
| | lysine) | poly(aspartic acid) | phosphatidyl | lactic acid) |
| | | | ethanolamine | |
| PNIPA-PBMA | PAA-PMMA | PEO-PPO-PEO | PEO-PCL | PEO-(C₁₆, BLA) |
| poly(N- | poly(acrylic acid)- | poly(ethylene | poly(ethylene | poly(ethylene |
| isopropylacrylamide)- | poly(methyl | oxide)- | oxide)-poly(E- | oxide)-C₁₆, (J3- |
| poly(butylmethacrylate) | methacrylate) | poly(propylene | caprolactone) | benzyl-L- |
| | | oxide)- | | aspartate) |
| | | poly(ethylene | | |
| | | oxide) | | |
| PEO-P(Asp, BLA) | LCC | | | |
| poly(ethylene oxide)- | N-laurel- | | | |
| poly(aspartic acid),(J3- | carboxymethyl- | | | |
| benzyl-L-aspartate) | chitosan | | | |

Suitable excipients for use in the composition of the present invention include, without limitation, any excipient which is relatively non-toxic and innocuous to a patient at concentrations consistent with effective activity of the solid dispersion of the invention so that any side effects ascribable to the excipients do not vitiate the beneficial effects of the solid dispersion. Such excipients include, without limitation, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired.

The amount of solid dispersion that may be included in a composition of the present invention is that amount which produces a result or exerts an influence on the particular individual receiving the solid dispersion. Solid dispersions of the present invention can be administered with, e.g., pharmaceutically-acceptable excipients well known in the art using any effective conventional dosage unit forms, including immediate, slow and timed release preparations, orally, parenterally, topically, nasally, ophthalmically, optically, sublingually, rectally, vaginally, and the like.

The composition comprising the solid dispersion may be used alone or in combination with other compositions to improve animal or human health or nutrition. Likewise, the solid dispersion itself may be combined with other compositions to improve animal health or nutrition.

The dispersions according to the present invention are suitable as dietary supplements, or as components in dietary supplements.

In some embodiments, the solid dispersion thereof is incorporated into a capsule.

In other embodiments, the solid dispersion thereof is incorporated into a tablet.

In yet other embodiments, the solid dispersion is added to a foodstuff. The solid dispersion may be added to any foodstuff, including, without limitation coffee, tea, soda, fruit drink, water, sauce, candy, cereal, bread, fruit mixes, fruit salads, salads, snack bars, fruit leather, health bars, granola, smoothies, soups, juices, cakes, pies, shakes, ice cream, health drinks.

EGCG can be used for prevention or therapy of various diseases or conditions, based on its antioxidant effects. It is useful for treatment or prevention of diseases including, but not limited to neurodegenerative diseases or conditions, such as Alzheimer's disease; upper respiratory diseases, including those caused by an infection; dementia, such as AIDS-dementia; oncological disorders, such as cancer; inflammatory or auto-immune diseases, such as rheumatoid arthritis or diabetic neuropathies; or a disease or condition caused by an infection by virus or bacteria.

The solid dispersions of the present invention may be administered in a single unit dosage form that contains an amount of solid dispersion effective to treat a subject. The solid dispersions can also include suitable excipients or stabilizers, and can be in solid or liquid form such as, tablets, capsules, powders, solutions, suspensions, or emulsions. Typically, the composition comprising the solid dispersion will contain from about 0.01 to 99% w/w, or from about 5 to 95% w/w solid dispersion.

The solid dispersion, when combined with any suitable excipients or stabilizers, and whether administered alone or in the form of a composition, can be administered orally. For most therapeutic purposes, the solid dispersion can be administered orally as a solid or as a solution or suspension in liquid form.

The solid unit dosage forms of the solid dispersion can be of a conventional type. The solid form can be a capsule, such as an ordinary gelatin type containing the solid dispersion and a carrier, for example, lubricants and inert fillers such as, lactose, sucrose, or cornstarch. In another embodiment, the solid dispersion is tableted with conventional tablet bases such as lactose, sucrose, or cornstarch in combination with binders like acacia or gelatin, disintegrating agents such as cornstarch, potato starch, or alginic acid, and a lubricant such as stearic acid or magnesium stearate.

The solid dispersion may be administered in combination with other therapeutic regimens that are known in the art, whether now known or hereafter developed.

### EXAMPLES

### Example 1

TABLE 4
Raw materials and vendors

| Material | Vendor |
|---|---|
| Teavigo ™ | Healthy |
| (150 mg/capsule, EGCG ≥ 94%) | Origins |
| NF Grade | Shin-Etsu |
| hydroxypropyl methylcellulose | Chemical |
| acetate succinate | |
| (HPMC-AS, MG) | |
| hydroxypropyl methylcellulose | Shin-Etsu |
| phthalate (HPMC-P, HP-55) | Chemical |
| Soluplus ® | BASF |
| cellulose acetate, average Mn ~30000 by GPC | Sigma-Aldrich |
| polyvinylpyrrolidone | Sigma-Aldrich |
| (PVT K-90), | |
| average Mw 360000 | |
| polyvinyl alcohol (PVA) | Sigma-Aldrich |
| polyvinyl acetate (PVAc) | Sigma-Aldrich |
| polyethylene glycol (PEG), average Mn 10000 | Sigma-Aldrich |
| Gelucire ® 50/13 | Gattefosse |
| dioxane | Sigma-Aldrich |
| water (HPLC grade) | Sigma-Aldrich |
| simulated gastric fluid (SGF) | RICCA Chemical |
| | Company |
| simulated intestinal fluid (SIF) | RICCA Chemical |
| | Company |

All chemicals were obtained from commercial sources and used without further purification unless otherwise indicated.

XRPD patterns were collected with a PANalytical X'Pert PRO MPD diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b. The data acquisition parameters for each pattern are displayed above the image in the Data section of this report including the divergence slit (DS) before the mirror and the incident-beam antiscatter slit (SS).

Polarized light microscopy (PLM) was performed using a Leica DM LP microscope with cross polarizer. SEM was performed using a FEI Quanta 200 scanning electron microscope equipped with an Everhart Thornley (ET) detector. Images were collected and analyzed using xTm (v. 2.01) and XT Docu (v. 3.2) software, respectively. The thermogravimetric analyses (TGA) were performed using a TA Instruments Q5000 IR thermogravimetric analyzer. Each sample was placed in an aluminum sample pan and inserted into the TGA furnace. The furnace was first equilibrated at 25° C., and then heated under N₂ at a rate of 10° C./min, up to a final temperature of 350° C. Modulated differential scanning calorimetry (mDSC) data were obtained on a TA Instruments Q2920 differential scanning calorimeter equipped with a refrigerated cooling system (RCS). The test was progressed by modulating temperature±0.8° C. every 60 seconds from 0 to 180° C. The reported glass transition temperature (T_{g}) is obtained from the inflection point of the step change in the reversing heat flow versus temperature curve.

### Example 2—Preparation of Raw EGCG

36 capsules of Teavigo were broken and the solids were suspended in ˜260 mL HPLC grade water and filtered to remove the solids. The resultant solution was clear with no evidence of solid particles observed. The solution was then dried under N₂ purge until water was fully removed based on visual observation. Such generated EGCG solids were directly used for generating amorphous EGCG and solid dispersions.

### Example 3—Screening for Dispersions

Nine polymers were investigated for performance in making solid dispersions with EGCG. For each polymer, individual solutions of polymer (200 mg in 3-10 mL dioxane depending on solubility) and EGCG (200 mg in 11.5 mL water) were prepared and then slowly mixed together with the procedures described generally in Example 4. The initial results are provided in Table 5. Upon mixing, precipitates were observed in the samples containing EGCG along with PVP, PVA, PEG and PVAc, and therefore these samples did not move forward with dispersion generation. Slight amount of precipitates were also noticed in the samples containing EGCG and other five polymers of HPMC-AS, HPMC-P, Soluplus®, cellulose acetate, and Gelucire® 50/13. These samples were clear again upon addition of more solvent and vortexing.

TABLE 5
Initial screen results of nine polymers with EGCG

| | Visual | Visual | |
|---|---|---|---|
| | observation | observation after | |
| Polymer | upon mixing | lyophilization | XRPD results |
| HPMC-AS | clear solution | light yellow | x-ray |
| | | fluffy particles | amorphous |
| HPMC-P | clear solution | yellow fluffy | x-ray |
| | | particles | amorphous |
| Soluplus ® | clear solution | white fluffy | x-ray |
| | | particles | amorphous |
| Cellulose | clear solution | light yellow | x-ray |
| acetate | | fluffy particles | amorphous |
| Gelucire ® | clear solution | light yellow | disordered |
| 50/13 | | fluffy particles | |
| PVA | flocculated | — | — |
| | suspension | | |
| PVP | flocculated | — | — |
| | suspension | | |
| PEG | flocculated | — | — |
| | suspension | | |
| PVAc | flocculated | — | — |
| | suspension | | |

These clear solutions containing EGCG and polymer were lyophilized to yield solids.

### Example 4—Preparation of Dispersions

EGCG solid dispersions were prepared by lyophilization using four polymers including HPMC-AS, HPMC-P, Soluplus®, and cellulose acetate. Individual solutions of polymer (in dioxane) and EGCG (in water) were prepared. The EGCG aqueous solution was added into the polymer-dioxane solution slowly and stepwisely, with each step about 1-2 mL of aqueous EGCG solution were added. During this step, additional fresh dioxane or water may be added into the samples followed by vigorous vortexing to reach a clear solution without visible solids or colloids. The approximate ratios of water to dioxane for the final solution of the dispersions are provided in Table 6 (per gram of EGCG).

TABLE 6
Final mixtures proportions of the four dispersions

| | EGCG (g) | Polymer (g) | H₂O (mL) | Dioxane (mL) |
|---|---|---|---|---|
| HPMC-AS | 1 | 1 | 50 | 80 |
| HPMC-P | 1 | 1 | 58 | 100 |
| Soluplus ® | 1 | 1 | 58 | 78 |
| Cellulose acetate | 1 | 1 | 58 | 140 |

For lyophilization, each EGCG/polymer final solution was frozen into a thin layer on the sides of the vial by rotating the sample vial in a cold bath of dry ice/acetone (at a temperature of −78° C.). The sample vial was then attached to a Flexi-Dry manifold lyophilizer (SP Industries, Stone Ridge, N.Y.) at −50° C. for 3 days. Pure EGCG aqueous solution was lyophilized as well to generate amorphous EGCG (aEGCG) as a reference material for comparison with the dispersions. After lyophilization was done, samples were secondary dried under vacuum at 40° C./5 days for dispersions, and under vacuum at RT/8 days for aEGCG, to remove the residual dioxane and water from samples. The lyophilized materials are fluffy and lightweight; the Soluplus® dispersion shows a white color while all the others (including the pure EGCG) display some yellowness. All dispersions were prepared at 50:50 (w/w, EGCG/polymer) composition.

### Example 5—Stress Tests at about 40° C./75% RH Stress

Solids of dispersion or amorphous EGCG (approximately 15˜30 mg each) were exposed to about 40° C./75% RH by placing solids into an uncapped clear glass vial inside a sealed container in a 40° C. oven. 75% RH were maintained by saturated salt solution of NaCL (ASTM Standard E, 104-85, 1996). Samples were stressed for 11 days, and then characterized by XRPD for evidence of crystallization.

### Example 6—Stress Tests in the GI Tract (Simulated)

Solids were suspended in media including simulated gastric fluid (SGF) and simulated intestinal fluid (SIF), and observed under PLM from time to time up to 24 hours for evidence of crystallization indicative by the appearance of birefringence/extinctions. The weights of the solids and the volumes of the media are listed in the following table. Because of the high solubility of aEGCG, more materials were suspended into the media compared with the dispersions.

TABLE 7
The amounts of the solids and media used for the stress testings

| | In SGF | | In SIF | |
|---|---|---|---|---|
| | Weight | SGF | Weight | SIF |
| | of solids | volume | of solids | volume |
| | (mg) | (μL) | (mg) | (μL) |
| aEGCG | 49.5 | 50 | 46.8 | 40 |
| EGCG/HPMC-AS dispersion | 4.6 | 100 | 4.4 | 100 |
| EGCG/HPMC-P dispersion | 3.5 | 100 | 4.2 | 100 |
| EGCG/Soluplus ® dispersion | 3.0 | 100 | 3.5 | 100 |
| EGCG/cellulose | 3.5 | 100 | 4.4 | 100 |
| acetate dispersion | | | | |

### Example 7—Dissolution Tests

Dissolution testing was performed in 100 mL of standard phosphate-buffered salines (pH 7.4) at 37° C. in a jacketed beaker. The temperature was precisely controlled by a Julabo Heating Circulator. EGCG concentration in the solution was in-situ monitored via an Ocean Optics USB2000+UV-VIS spectrometer equipped with an RT-2MM Dip Probe. The testing was performed with continuous stirring until all solids were dissolved and the medium was free of observable particles, or up to 24 hours. For early period of dissolution, the data acquisition density was relatively large to capture all sudden changes; while at later periods, the data acquisition frequency was reduced due to slow evolution in the medium. For each material, an equivalency of 20 mg EGCG was tested and the experiment was monitored until all solids were dissolved or up to 24 hours. Based on the dissolution data, all EGCG (˜20 mg) were released into the medium within the period of dissolution tests for all samples. The actual amounts of materials tested are listed in following table.

TABLE 8
Weight of each sample used for dissolution test

| Material | Weight of sample (mg) |
|---|---|
| cEGCG | 20.3 |
| aEGCG | 23.0 |
| EGCG/HPMC-AS dispersion | 46.7 |
| EGCG/HPMC-P dispersion | 47.3 |
| EGCG/Soluplus ® dispersion | 42.7 |
| EGCG/cellulose acetate dispersion | 45.3 |

### Example 8—Stability Studies on Dispersions of Varying Weight Rates

20 capsules of Teavigo™ were broken and the solids were suspended in ˜300 mL HPLC grade water and filtered to remove the solids. The resultant solution was clear with no evidence of solid particles observed. The solution was then dried under N₂ purge until water is fully removed based on visual observation. Such generated EGCG solids were directly used for generating amorphous EGCG and solid dispersions.

EGCG solid dispersions were prepared by lyophilization using four polymers including HPMC-AS, HPMC-P, Soluplus®, and cellulose acetate. Individual solutions of polymer (in dioxane) and EGCG (in water) were prepared. The EGCG aqueous solution was added into the polymer-dioxane solution slowly. Additional fresh dioxane were added into the samples followed by vigorous vortexing to reach a clear solution without visible solids or colloids. The approximate volumes of water and dioxane for the final solution of the dispersions are provided in Table 9 (per gram of EGCG).

TABLE 9
The volumes of H₂O and dioxane per gram of EGCG
in the final mixtures for lyophilization

| | H₂O | Dioxane |
|---|---|---|
| | (mL) | (mL) |
| 91:9 (w/w) EGCG/Soluplus ® dispersion | 63.1 | 65.4 |
| 83:17 (w/w) EGCG/Soluplus ® dispersion | 65.5 | 76.0 |
| 67:33 (w/w) EGCG/Soluplus ® dispersion | 61.9 | 92.9 |
| 50:50 (w/w) EGCG/Soluplus ® dispersion | 62.0 | 134.3 |
| 9:91 (w/w) EGCG/Soluplus ® dispersion | 66.0 | 707.5 |
| 91:9 (w/w) EGCG/HPMC-AS dispersion | 64.0 | 104.2 |
| 83:17 (w/w) EGCG/HPMC-AS dispersion | 57.3 | 110.7 |
| 67:33 (w/w) EGCG/HPMC-AS dispersion | 61.7 | 150.6 |
| 91:9 (w/w) EGCG/HPMC-P dispersion | 59.5 | 96.9 |
| 83:17 (w/w) EGCG/HPMC-P dispersion | 59.9 | 115.8 |
| 67:33 (w/w) EGCG/HPMC-P dispersion | 54.8 | 164.4 |
| 91:9 (w/w) EGCG/ cellulose acetate dispersion | 61.3 | 90.5 |
| 83:17 (w/w) EGCG/cellulose acetate dispersion | 62.1 | 126.3 |
| 67:33 (w/w) EGCG/cellulose acetate dispersion | 62.3 | 162.1 |

For lyophilization, each EGCG/polymer solution was frozen in a cold bath of dry ice/acetone (at a temperature of −78° C.). The sample vial was then kept in a lyophilizer at −30° C. for 1 day, −10° C. for 2 days, 0° C. for 1 day, and then 20° C. for 4 hours. Pure EGCG aqueous solution was lyophilized as well to generate amorphous EGCG (EGCG) as a reference material for comparison with the dispersions. The lyophilized materials are free flowing materials. Table 10 shows the color results, based on visual observation, before and after such stressing.

TABLE 10
Color Observations Before and After Stress

| | As | After |
|---|---|---|
| Material | generated | stress |
| 91:9 (w/w) EGCG/ | very light pink, | dark red |
| Soluplus ® dispersion | off white | |
| 83:17 (w/w) EGCG/ | very light pink, | dark red |
| Soluplus ® dispersion | off white | |
| 67:33 (w/w) EGCG/ | very light pink, | dark red |
| Soluplus ® dispersion | off white | |
| 50:50 (w/w) EGCG/ | very light pink, | light pink |
| Soluplus ® dispersion | off white | |
| 9:91 (w/w) EGCG/ | very light pink, | light yellow |
| Soluplus ® dispersion | off white | |
| 91:9 (w/w) EGCG/ | very light pink, | very dark red |
| HPMC-AS dispersion | off white | |
| 83:17 (w/w) EGCG/ | very light pink, | very dark red |
| HPMC-AS dispersion | off white | |
| 67:33 (w/w) EGCG/ | very light pink, | very dark red |
| HPMC-AS dispersion | off white | |
| 91:9 (w/w) EGCG/ | very light pink, | very dark red |
| HPMC-P dispersion | off white | |
| 83:17 (w/w) EGCG/ | very light pink, | very dark red |
| HPMC-P dispersion | off white | |
| 67:33 (w/w) EGCG/ | very light pink, | very dark red |
| HPMC-P dispersion | off white | |
| 91:9 (w/w) EGCG/ | very light pink, | very dark red |
| cellulose acetate dispersion | off white | |
| 83:17 (w/w) EGCG/ | very light pink, | very dark red |
| cellulose acetate dispersion | off white | |
| 67:33 (w/w) EGCG/ | very light pink, | very dark red |
| cellulose acetate dispersion | off white | |
| amorphous EGCG | very light pink, | pink |
| | off white | |

The following clauses provide numerous embodiments and are non-limiting:

Clause 1. A solid dispersion comprising amorphous EGCG and a polymer.

Clause 2. The solid dispersion of clause 1, wherein the polymer contains a cellulose functionality.

Clause 3. The solid dispersion of clause 1, wherein the polymer contains a caprolactam functionality.

Clause 4. The solid dispersion of clause 2, wherein the polymer is selected from the group consisting of HPMC-AS, HPMC-P, and cellulose acetate.

Clause 5. The solid dispersion of clause 4, wherein the polymer is HPMC-AS.

Clause 6. The solid dispersion of clause 4, wherein the polymer is HPMC-P.

Clause 7. The solid dispersion of clause 4, wherein the polymer is cellulose acetate.

Clause 8. The solid dispersion of clause 3, wherein the polymer is polyvinyl caprolactam-polyvinyl acetate-polyethylene glycol graft co-polymer.

Clause 9. The solid dispersion of clause 8, wherein the polymer is Soluplus®.

Clause 10. The solid dispersion of clauses 8 or 9, wherein the weight ratio of amorphous EGCG to polymer is between about 1:10 and about 10:1.

Clause 11. The solid dispersion of clauses 3 or 4, wherein the weight ratio of amorphous EGCG to polymer is about 1:1.

Clause 12. The solid dispersion of clause 10, wherein the weight ratio of amorphous EGCG to polymer is about 1:1.

Clause 13. A pharmaceutical composition comprising a solid-dispersion of clauses 1-12.

Clause 14. The pharmaceutical composition of clause 13, further comprising one or more pharmaceutically acceptable excipients.

Clause 15. A method of sustained release delivery of EGCG comprising administering a pharmaceutical composition of clauses 13 or 14 to a human.

Clause 16. A sustained release pharmaceutical composition comprising a solid dispersion of clauses 1-12.

Clause 17. The solid dispersion of clause 5, wherein the glass transition temperature is less than about 80° C.

Clause 18. The solid dispersion of clause 17, wherein the glass transition temperature is about 72° C.

Clause 19. The solid dispersion of clauses 8 or 9 wherein the glass transition temperature is less than about 150° C.

Clause 20. The solid dispersion of clause 19, wherein the glass transition temperature is about 143° C.

Clause 21. The solid dispersion of clause 5, wherein the x-ray powder diffraction pattern is x-ray amorphous after being stressed at 40° C. and 75% relative humidity after 11 days.

Clause 22. The solid dispersion of clauses 1-14, wherein the EGCG released within 20 minutes is less than that of amorphous EGCG in a pH 7.4 PBS medium at 37° C.

Clause 23. The solid dispersion of clauses 1-14, wherein the EGCG released within 20 minutes is less than that of crystalline EGCG in a pH 7.4 PBS medium at 37° C.

Clause 24. The solid dispersion of clauses 1, 3, 8, or 9 wherein less than 60% of the EGCG is released within 20 minutes.

Clause 25. The solid dispersion of clause 24, wherein about 50% of the EGCG is released within 20 minutes.

Clause 26. A solid dispersion comprising EGCG and a micelle-forming polymer.

Clause 27. The dispersion of clause 26, wherein the dispersion is selected from PEO-PBLA, PEO-P(Lys), PEO-P(Asp), PEO-PE, PEO-PDLLA, PNIPA-PBMA, PAA-PMMA, PEO-PPO-PEO, PEO-PCL, PEO-(C16,BLA), PEO-P(Asp,BLA), and LCC.

Clause 28. A supplement comprising a solid-dispersion of clauses 1-12.

Clause 29. A method of delivering EGCG comprising administering a pharmaceutical composition of clauses 13 or 14 to a human.

Clause 30. The pharmaceutical composition of clauses 13, 14, 16 in tablet or capsule form.

Clause 31. A foodstuff additive comprising a solid dispersion of EGCG and a polymer.

Clause 32. The foodstuff additive of clause 31, wherein the polymer is selected from the group consisting of HPMC-AS, HPMC-P, cellulose acetate, and a polyvinyl caprolactam-polyvinyl acetate-polyethylene glycol graft co-polymer.

Clause 33. The foodstuff additive of clause 31, wherein the polymer is Soluplus®.

1. A solid dispersion comprising amorphous EGCG and a polymer.

2. The solid dispersion of claim 1, wherein the polymer contains a cellulose functionality.

3. The solid dispersion of claim 1, wherein the polymer contains a caprolactam functionality.

4. The solid dispersion of claim 2, wherein the polymer is selected from the group consisting of HPMC-AS, HPMC-P, and cellulose acetate.

5. The solid dispersion of claim 4, wherein the polymer is HPMC-AS.

6. The solid dispersion of claim 4, wherein the polymer is HPMC-P.

7. The solid dispersion of claim 4, wherein the polymer is cellulose acetate.

8. The solid dispersion of claim 3, wherein the polymer is polyvinyl caprolactam-polyvinyl acetate-polyethylene glycol graft co-polymer.

9. The solid dispersion of claim 8, wherein the polymer is Soluplus®.

10. The solid dispersion of claim 8, wherein the weight ratio of amorphous EGCG to polymer is between about 1:10 and about 10:1.

11. The solid dispersion of claim 3, wherein the weight ratio of amorphous EGCG to polymer is about 1:1.

12. The solid dispersion of claim 10, wherein the weight ratio of amorphous EGCG to polymer is about 1:1.

13. A pharmaceutical composition comprising the solid-dispersion of claim 1.

14. The pharmaceutical composition of claim 13, further comprising one or more pharmaceutically acceptable excipients.

15. A method of sustained release delivery of EGCG comprising:
administering a pharmaceutical composition of claim 13 to a human.

16. A sustained release pharmaceutical composition comprising the solid dispersion of claim 1.

17. The solid dispersion of claim 5, wherein the glass transition temperature is less than about 80° C.

18. The solid dispersion of claim 17, wherein the glass transition temperature is about 72° C.

19. The solid dispersion of claim 8, wherein the glass transition temperature is less than about 150° C.

20. The solid dispersion of claim 19, wherein the glass transition temperature is about 143° C.

21. The solid dispersion of claim 5, wherein the x-ray powder diffraction pattern is x-ray amorphous after being stressed at about 40° C. and 75% relative humidity after 11 days.

22. The solid dispersion of claim 1, wherein the EGCG released within 20 minutes is less than that of amorphous EGCG in a pH 7.4 PBS medium at 37° C.

23. The solid dispersion of claim 1, wherein the EGCG released within 20 minutes is less than that of crystalline EGCG in a pH 7.4 PBS medium at 37° C.

24. The solid dispersion of claim 1, wherein less than 60% of the EGCG is released within 20 minutes.

25. The solid dispersion of claim 24, wherein about 50% of the EGCG is released within 20 minutes.

26. A solid dispersion comprising EGCG and a micelle-forming polymer.

27. The dispersion of claim 26, wherein the dispersion is selected from PEO-PBLA, PEO-P(Lys), PEO-P(Asp), PEO-PE, PEO-PDLLA, PNIPA-PBMA, PAA-PMMA, PEO-PPO-PEO, PEO-PCL, PEO-(C16,BLA), PEO-P(Asp,BLA), and LCC.

28. A supplement comprising the solid-dispersion of claim 1.

29. A method of delivering EGCG comprising:
administering a pharmaceutical composition of claim 13 to a human.

30. The pharmaceutical composition of claim 13 in tablet or capsule form.

31. A foodstuff additive comprising a solid dispersion of EGCG and a polymer.

32. The foodstuff additive of claim 31, wherein the polymer is selected from the group consisting of HPMC-AS, HPMC-P, cellulose acetate, and a polyvinyl caprolactam-polyvinyl acetate-polyethylene glycol graft co-polymer.

33. The foodstuff additive of claim 31, wherein the polymer is Soluplus®
